(19) United States
(12) Patent Application Publication
(10) Pub. No.: US 2023/0151398 A1
(43) Pub. Date: May 18, 2023

(54) MODIFIED MICROORGANISM AND METHOD FOR THE IMPROVED PRODUCTION OF ECTOINE

(71) Applicant: METABOLIC EXPLORER, SAINT BEAUZIRE (FR)

(72) Inventors: Laurence DUMON-SEIGNOVERT, PONT DU CHATEAU (FR); Céline RAYNAUD, SAINT BEAUZIRE (FR); Thomas DESFOUGERES, DISSAY (FR)

(73) Assignee: METABOLIC EXPLORER, SAINT BEAUZIRE (FR)

(21) Appl. No.: 17/797,907

(22) PCT Filed: Feb. 8, 2021
(86) PCT No.: PCT/EP2021/052973

§ 371 Date: Aug. 5, 2022

(30) Foreign Application Priority Data
Feb. 7, 2020 (WO) 20305122.2

(51) Int. Cl. C12P 17/12; C12N 9/10

(52) U.S. Cl. CPC C12P 17/12; C12N 9/1025; C12Y 203/01178; C12Y 203/03001

(57) ABSTRACT

The present invention relates to a microorganism genetically modified for production of ectoine, wherein said microorganism comprises the following modifications: expression of a heterologous gene ectA encoding a diaminobutyric acid acetyltransferase having at least 90% similarity with SEQ ID NO: 1, SEQ ID NO: 2, SEQ ID NO: 3, SEQ ID NO: 4 or SEQ ID NO: 5, a heterologous gene ectB encoding a diaminobutyric acid aminotransferase having at least 90% similarity with SEQ ID NO: 6, SEQ ID NO: 7, SEQ ID NO: 8, SEQ ID NO: 9 or SEQ ID NO: 10, a heterologous gene ectC encoding an ectoine synthase having at least 90% similarity with SEQ ID NO: 11, SEQ ID NO: 12, SEQ ID NO: 13, SEQ ID NO: 14 or SEQ ID NO: 15 and deletion of pykA and pykF genes. The present invention also relates to a method for the production of ectoine using said microorganism.

### FIELD OF THE INVENTION

The present invention relates to a microorganism genetically modified for the improved production of ectoine and to a method for the improved production of ectoine using said microorganism.

### BACKGROUND OF THE INVENTION

Ectoine, also known as 1,4,5,6-tetrahydro-2-methyl-4-pyrimidinecarboxylic acid or (S)-2-methyl-3,4,5,6-tetrahydropyrimidine-4-carboxylic acid, is a polar, soluble, uncharged cyclic amino acid derivative. First discovered in the halophilic microorganism Halorhodospira halochloris, ectoine functions as an osmolyte, protecting membranes, enzymes, nucleic acids, etc. from high osmotic stress/dehydration. It has also been found to protect cells against other stresses including UV radiation, heating, freezing, and chemical agents. In view of its protective effect, ectoine has a wide variety of commercial applications in cosmetics, oral and skin care products, and as a cryoprotectant or protein stabilizer (Goraj et al., 2019). Ectoine is also of interest in the pharmaceutical industry in view of its potential therapeutic applications, in particular its use in the treatment of inflammation or neurodegenerative disease. Production of ectoine is estimated at approximately 15 tons per year worldwide, with ectoine representing a valuable commodity, retailing at an estimated $1000/kg (Strong et al., 2016).

In halophilic microorganisms, ectoine is synthesized from the L-aspartate-β-semialdehyde (ASA) precursor in three steps with the enzymes EctA, EctB, and EctC. The diaminobutyric acid transaminase (EctB, also referred to as a diaminobutyric acid aminotransferase) initially converts ASA to L-2,4-diaminobutyric acid (DABA). DABA is then converted to Nγ-acetyl-L-2,4-diaminobutyric acid by the DABA acetyltransferase (EctA). Finally, ectoine synthase (EctC) converts Nγ-acetyl-L-2,4-diaminobutyric acid to ectoine. Genes encoding these enzymes are organized in either ectABC or ectABC-ask operons with ask coding for an aspartokinase, and are generally expressed in response to high salt conditions and temperature extremes.

While ectoine may be chemically synthesized (see, e.g., Goraj et al., 2019, JPH0331265, WO 2010/006792), this is not competitive with microbial processes due to the high cost of precursors, such as DABA, reaction complexity and low stereo-specificity of synthesized ectoine. In contrast, processes using microorganisms having the appropriate metabolic pathway represent a sustainable way to produce ectoine at a lower cost. Indeed, not only are such processes generally more environmentally friendly, enzyme stereo-selectivity leads to the production of L-ectoine only.

Microbial production processes are mainly based on so-called “bacterial milking” in which halophilic bacteria, such as Halomonas elongata or Chromohalobacter salexigens, are initially cultivated in conditions of high salinity (e.g., 10-20% NaCl), inducing ectoine production, and then subjected to a hypoosmotic shock (e.g., 2% NaCl), causing cytoplasmic ectoine to be released into the culture medium. Ectoine titers obtained based on this technique have been reported as ranging from 6.04 g/L to 32.9 g/L (Rui-Feng et al., 2017, Fallet et al., 2010). However, high salt levels inevitably cause equipment corrosion and increase the complexity of downstream processing as the recovered product must be desalted, which in turn increases cost. Alternating between high and low salt conditions furthermore results in discontinuous production of ectoine. Yield may also be reduced in such bacteria due to their ability to catabolize ectoine, reusing it as a source of carbon.

Genetic modification of non-halophilic bacteria, such as Corynebacterium glutamicum or Escherichia coli, to produce ectoine has also been described (Becker et al., 2013, Schubert et al., 2007, He et al., 2015, Ning et al., 2016). Decoupling ectoine production from osmolarity in these bacteria eliminates the need for high salt conditions. In addition to expressing the ectABC genes for ectoine production, the following additional genetic modifications may be incorporated: deletion of thrA and iciR, and introduction of the heterologous feedback-resistant gene, lysC. However, ectoine yield and productivity remain equivalent than that obtained in halophilic bacteria (e.g., ranging from 5.4 g/L as described in Schubert et al., 2007 to 25.1 g/L in E. coli as described in Ning et al., 2016).

Thus, there remains a need for improved microorganisms that are able to produce ectoine, in particular in low-salt conditions. There also remains a need for cost-effective, simple, and rapid methods for producing ectoine, ideally wherein the production, titer, and/or yield of ectoine is improved when compared to currently used methods involving halophilic bacteria.

### BRIEF DESCRIPTION OF THE INVENTION

The present invention addresses the above needs, providing a microorganism genetically modified for the improved production of ectoine and a simple, rapid method of producing ectoine on an industrial scale using said microorganism. The microorganism genetically modified for the production of ectoine provided herein notably comprises the following modifications:
expression of
   heterologous gene ectA encoding a diaminobutyric acid acetyltransferase having at least 90% similarity with SEQ ID NO: 1, SEQ ID NO: 2, SEQ ID NO: 3, SEQ ID NO: 4 or SEQ ID NO: 5,
   heterologous gene ectB encoding a diaminobutyric acid aminotransferase having at least 90% similarity with SEQ ID NO: 6, SEQ ID NO: 7, SEQ ID NO: 8, SEQ ID NO: 9 or SEQ ID NO: 10,
   heterologous gene ectC encoding an ectoine synthase having at least 90% similarity with SEQ ID NO: 11, SEQ ID NO: 12, SEQ ID NO: 13, SEQ ID NO: 14 or SEQ ID NO: 15,
and
deletion of pykA and pykF genes.

Indeed, the inventors have surprisingly and unexpectedly found that the production of ectoine can be improved by deleting the pykA and pykF genes, coding for the two major pyruvate kinase isoenzymes, in microorganisms genetically modified to produce ectoine.

Preferably, the microorganism further comprises at least a 50% reduction in citrate synthase enzyme activity as compared to an unmodified microorganism, more preferably at least a 75% reduction in citrate synthase activity.

Preferably, said citrate synthase enzyme has at least 90% similarity with the citrate synthase enzyme of SEQ ID NO: 18, SEQ ID NO: 19, SEQ ID NO: 20, or SEQ ID NO: 21, encoded by the gene gltA.

Preferably, the expression of the gltA gene, encoding said citrate synthase enzyme having at least 90% similarity with the citrate synthase enzyme of SEQ ID NO: 18, SEQ ID NO: 19, SEQ ID NO: 20, or SEQ ID NO: 21, is under the control of promoter PgltA or under the control of a heterologous inducible promoter, said promoter preferably being selected from the group consisting of a trc promoter (Ptrc), a tac promoter (Ptac), a lac promoter (Plac), a tet promoter (Ptet), a lambda P_{L} promoter (PL), and a lambda P_{R} promoter (PR).

Preferably, the microorganism further comprises a deletion of the gene ppc encoding the phosphoenol pyruvate carboxylase and an overexpression of the gene pck encoding a phosphoenolpyruvate carboxykinase having at least 90% similarity with SEQ ID NO: 29, SEQ ID NO: 30, SEQ ID NO: 31, SEQ ID NO: 32 or SEQ ID NO: 33.

Preferably, the microorganism further comprises overexpression of an aspartate transaminase having at least 90% similarity with SEQ ID NO: 35, SEQ ID NO: 36, SEQ ID NO: 37, or SEQ ID NO: 38, and a glutamate dehydrogenase having at least 90% similarity with SEQ ID NO: 39, SEQ ID NO: 40, SEQ ID NO: 41, SEQ ID NO: 42, SEQ ID NO: 43, or SEQ ID NO: 44.

Preferably, the microorganism further comprises a deletion of at least one gene selected from the group consisting of ackA-pta, adhE, frdABCD, IdhA, mgsA, pflAB, and mdh.

Preferably, the microorganism has been genetically modified to be able to utilize sucrose as a carbon source, more preferably wherein said microorganism further comprises the overexpression of:
the heterologous cscBKAR genes of E. coli EC3132, or
the heterologous scrKYABR genes of Salmonella sp.

Preferably, the microorganism belongs to the family of bacteria Enterobacteriaceae, Clostridiaceae, Bacillaceae, Streptomycetaceae, or Corynebacteriaceae, or to the family of yeasts Saccharomycetaceae. More preferably, said Enterobacteriaceae bacterium is Escherichia coli or Klebsiella pneumoniae, said Clostridiaceae bacterium is Clostridium acetobutylicum, said Corynebacteriaceae bacterium is Corynebacterium glutamicum, or said Saccharomycetaceae yeast is Saccharomyces cerevisiae. Even more preferably, said Enterobacteriaceae bacterium is Escherichia coli.

The invention further relates to a method for the production of ectoine comprising the steps of:
a) culturing a microorganism genetically modified for the production of ectoine as provided herein in an appropriate culture medium comprising a source of carbon and a source of nitrogen, and
b) recovering ectoine from said culture medium.

Preferably, the source of carbon is glycerol and/or glucose and/or sucrose.

Preferably, step b) comprises a step of filtration, desalination, cation exchange, liquid extraction, or distillation.

### DETAILED DESCRIPTION OF THE INVENTION

Before describing the present invention in detail, it is to be understood that the invention is not limited to particularly exemplified microorganism and/or methods and may, of course, vary. Indeed, various modifications, substitutions, omissions, and changes may be made without departing from the scope of the invention. It shall also be understood that the terminology used herein is for the purpose of describing particular embodiments of the invention only, and is not intended to be limiting.

All publications, patents and patent applications cited herein, whether supra or infra, are hereby incorporated by reference in their entirety. Furthermore, the practice of the present invention employs, unless otherwise indicated, conventional microbiological and molecular biological techniques that are within the skill of the art. Such techniques are well-known to the skilled person, and are fully explained in the literature.

Unless defined otherwise, all technical and scientific terms used herein have the same meanings as are commonly understood by one of ordinary skill in the art to which this invention belongs. Although any materials and methods similar or equivalent to those described herein can be used to practice or test the present invention, preferred material and methods are provided.

It must be noted that as used herein and in the appended claims, the singular forms “a,” “an,” and “the,” include plural reference unless the context clearly dictates otherwise. Thus, for example, a reference to “a microorganism” includes a plurality of such microorganisms, and a reference to “an endogenous gene” is a reference to one or more endogenous genes, and so forth.

The terms “comprise,” “contain,” and “include” and variations thereof such as “comprising” are used herein in an inclusive sense, i.e., to specify the presence of the stated features but not to preclude the presence or addition of further features in various embodiments of the invention.

A first aspect of the invention concerns a microorganism genetically modified for the improved production of ectoine.

The term “microorganism,” as used herein, refers to a living microscopic organism, which may be a single cell or a multicellular organism and which can generally be found in nature. In the present context, the microorganism is preferably a bacterium, yeast or fungus. Preferably, the microorganism of the invention is selected from the Enterobacteriaceae, Clostridiaceae, Bacillaceae, Streptomycetaceae, or Corynebacteriaceae family or from among yeast, more preferably from the Saccharomycetaceae family. Even more preferably, the microorganism of the invention is a species of Escherichia, Klebsiella, Thermoanaerobacterium, Clostridium, Corynebacterium or Saccharomyces. Even more preferably, said Enterobacteriaceae bacterium is Escherichia coli or Klebsiella pneumoniae, said Clostridiaceae bacterium is Clostridium acetobutylicum, said Corynebacteriaceae bacterium is Corynebacterium glutamicum, or said Saccharomycetaceae yeast is Saccharomyces cerevisiae. Most preferably, the microorganism of the invention is Escherichia coli.

The terms “recombinant microorganism” or “microorganism genetically modified” are used interchangeably herein and refer to a microorganism or a strain of microorganism that has been genetically modified or genetically engineered. This means, according to the usual meaning of these terms, that the microorganism of the invention is not found in nature and is genetically modified when compared to the “parental” microorganism from which it is derived. The “parental” microorganism may occur in nature (i.e., a wild-type microorganism) or may have been previously modified. The recombinant microorganisms of the invention may notably be modified by the introduction, deletion and/or modification of genetic elements. Such modifications can be performed, for example, by genetic engineering or by adaptation, wherein a microorganism is cultured in conditions that apply a specific stress on the microorganism and induce mutagenesis.

A microorganism genetically modified for the production of ectoine means that said microorganism is a recombinant microorganism as defined herein that is capable of producing ectoine. In other words, said microorganism has been genetically modified to allow production of ectoine.

A microorganism may notably be modified to modulate the expression level of an endogenous gene. The term “endogenous gene” means that the gene was present in the microorganism before any genetic modification. Endogenous genes may be overexpressed by introducing heterologous sequences in addition to, or to replace, endogenous regulatory elements. Endogenous genes may also be overexpressed by introducing one or more supplementary copies of the gene into the chromosome or on a plasmid. In this case, the endogenous gene initially present in the microorganism may be deleted. Endogenous gene expression levels, protein expression levels, or the activity of the encoded protein, can also be increased or attenuated by introducing mutations into the coding sequence of a gene or into non-coding sequences. These mutations may be synonymous, when no modification in the corresponding amino acid occurs, or non-synonymous, when the corresponding amino acid is altered. Synonymous mutations do not have any impact on the function of translated proteins, but may have an impact on the regulation of the corresponding genes or even of other genes, if the mutated sequence is located in a binding site for a regulator factor. Non-synonymous mutations may have an impact on the function or activity of the translated protein as well as on regulation depending the nature of the mutated sequence.

In particular, mutations in non-coding sequences may be located upstream of the coding sequence (i.e., in the promoter region, in an enhancer, silencer, or insulator region, in a specific transcription factor binding site) or downstream of the coding sequence. Mutations introduced in the promoter region may be in the core promoter, proximal promoter or distal promoter. Mutations may be introduced by site-directed mutagenesis using, for example, Polymerase Chain Reaction (PCR), by random mutagenesis techniques for example via mutagenic agents (Ultra-Violet rays or chemical agents like nitrosoguanidine (NTG) or ethylmethanesulfonate (EMS)) or DNA shuffling or error-prone PCR or using culture conditions that apply a specific stress on the microorganism and induce mutagenesis. The insertion of one or more supplementary nucleotide(s) in the region located upstream of a gene can notably modulate gene expression.

A particular way of modulating endogenous gene expression is to exchange the endogenous promoter of a gene (e.g., wild-type promoter) with a stronger or weaker promoter to upregulate or downregulate expression of the endogenous gene. The promoter may be endogenous (i.e., originating from the same species) or exogenous (i.e., originating from a different species). It is well within the ability of the person skilled in the art to select an appropriate promoter for modulating the expression of an endogenous gene. Such a promoter be, for example, a Ptrc, Ptac, Ptet, or Plac promoter, or a lambda P_{L} (PL) or lambda P_{R} (PL) promoter. The promoter may be “inducible” by a particular compound or by specific external conditions, such as temperature or light or a small molecule, such as an antibiotic.

A microorganism may also be genetically modified to express one or more exogenous or heterologous genes so as to overexpress the corresponding gene product (e.g., an enzyme). The terms “exogenous gene” or “heterologous gene” are used interchangeably herein and indicate that a gene was introduced into a microorganism wherein said gene is not naturally occurring in said microorganism. The genes ectA, ectB, and ectC are notably heterologous genes in the context of the present invention. In particular, the exogenous gene may be directly integrated into the chromosome of the microorganism, or be expressed extra-chromosomally within the microorganism by plasmids or vectors. For successful expression, the exogenous gene(s) must be introduced into the microorganism with all of the regulatory elements necessary for their expression or be introduced into a microorganism that already comprises all of the regulatory elements necessary for their expression. The genetic modification or transformation of microorganisms with one or more exogenous genes is a routine task for those skilled in the art.

One or more copies of a given exogenous gene can be introduced on a chromosome by methods well-known in the art, such as by genetic recombination. When a gene is expressed extra-chromosomally, it can be carried by a plasmid or a vector. Different types of plasmid are notably available, which may differ in respect to their origin of replication and/or on their copy number in the cell. For example, a microorganism transformed by a plasmid can contain 1 to 5 copies of the plasmid, about 20 copies, or even up to 500 copies, depending on the nature of the selected plasmid. A variety of plasmids having different origins of replication and/or copy numbers are well-known in the art and can be easily selected by the skilled practitioner for such purposes, including, for example, pTrc, pACYC184, pBR322, pUC18, pUC19, pKC30, pRep4, pHS1, pHS2, or pPLc236.

It should be understood that, in the context of the present invention, when an exogenous gene encoding a protein of interest is expressed in a microorganism, such as a non-halophilic microorganism (e.g., E. coli), a synthetic version of this gene may preferably be constructed by replacing non-preferred codons or less preferred codons with preferred codons of said microorganism which encode the same amino acid. Indeed, it is well-known in the art that codon usage varies between microorganism species, and that this may impact the recombinant expression level of a protein of interest. To overcome this issue, codon optimization methods have been developed, and are extensively described by Graf et al. (2000), Deml et al. (2001) and Davis & Olsen (2011). Several software programs have notably been developed for codon optimization determination such as the GeneOptimizer® software (Lifetechnologies) or the OptimumGene™ software of (GenScript). In other words, the exogenous gene encoding a protein of interest is preferably codon-optimized for expression in the chosen microorganism.

On the basis of a given amino acid sequence, the skilled person is furthermore able to identify an appropriate polynucleotide coding for said polypeptide (e.g., in the available databases, such as Uniprot), or to synthesize the corresponding polypeptide or a polynucleotide coding for said polypeptide. De novo synthesis of a polynucleotide can be performed, for example, by initially synthesizing individual nucleic acid oligonucleotides and hybridizing these with oligonucleotides complementary thereto, such that they form a double-stranded DNA molecule, and then ligating the individual double-stranded oligonucleotides such that the desired nucleic acid sequence is obtained.

The terms “expressing,” “overexpressing,” or “overexpression” of a protein of interest, such as an enzyme, refer herein to an increase in the expression level and/or activity of said protein in a microorganism, as compared to the corresponding parent microorganism that does not comprise the modification present in the genetically modified microorganism. A heterologous gene/protein can be considered to be “expressed” or “overexpressed” in a genetically modified microorganism when compared with a corresponding parent microorganism in which said heterologous gene/protein is absent. In contrast, the terms “attenuating” or “attenuation” of a protein of interest refer to a decrease in the expression level and/or activity of said protein in a microorganism, as compared to the parent microorganism. The attenuation of expression can notably be due to either the exchange of the wild-type promoter for a weaker natural or synthetic promoter or the use of an agent reducing gene expression, such as antisense RNA or interfering RNA (RNAi), and more particularly small interfering RNAs (siRNAs) or short hairpin RNAs (shRNAs). Promoter exchange may notably be achieved by the technique of homologous recombination (Datsenko & Wanner, 2000). The complete attenuation of the expression level and/or activity of a protein of interest means that expression and/or activity is abolished; thus, the expression level of said protein is null. The complete attenuation of the expression level and/or activity of a protein of interest may be due to the complete suppression of the expression of a gene. This suppression can be either an inhibition of the expression of the gene, a deletion of all or part of the promoter region necessary for expression of the gene, or a deletion of all or part of the coding region of the gene. A deleted gene can notably be replaced by a selection marker gene that facilitates the identification, isolation and purification of the modified microorganism. As a non-limiting example, suppression of gene expression may be achieved by the technique of homologous recombination, which is well-known to the person skilled in the art (Datsenko & Wanner, 2000).

Modulating the expression level of one or more proteins may thus occur by altering the expression of one or more endogenous genes that encode said protein within the microorganism as described above or by introducing one or more heterologous genes that encode said protein into the microorganism.

The term “expression level” as used herein, refers to the amount (e.g., relative amount, concentration) of a protein of interest (or of the gene encoding said protein) expressed in a microorganism, which is measurable by methods well-known in the art. The level of gene expression can be measured by various known methods including Northern blotting, quantitative RT-PCR, and the like. Alternatively, the level of expression of the protein coded by said gene may be measured, for example by SDS-PAGE, HPLC, LC/MS and other quantitative proteomic techniques (Bantscheff et al., 2007), or, when antibodies against said protein are available, by Western Blot-Immunoblot (Burnette, 1981), Enzyme-linked immunosorbent assay (e.g., ELISA) (Engvall and Perlman, 1971), protein immunoprecipitation, immunoelectrophoresis, and the like. The copy number of an expressed gene can be quantified, for example, by restricting chromosomal DNA followed by Southern blotting using a probe based on the gene sequence, fluorescence in situ hybridization (FISH), RT-qPCR, and the like.

Overexpression of a given gene or the corresponding protein may be verified by comparing the expression level of said gene or protein in the genetically modified organism to the expression level of the same gene or protein in a control microorganism that does not have the genetic modification (i.e., the parental strain).

The microorganism genetically modified for the production of ectoine provided herein comprises a heterologous enzyme having diaminobutyric acid acetyltransferase activity, a heterologous enzyme having diaminobutyric acid aminotransferase activity, and a heterologous enzyme having ectoine synthase activity, and is attenuated for pyruvate kinase activity of PykA and PykF. Preferably, when the activity of the PykA and PykF enzymes is attenuated, said activity is completely attenuated. Complete attenuation is preferably due to a partial or complete deletion of the gene coding for said enzyme, more preferably a complete deletion of the pykA and pykF genes coding for said enzymes.

The terms “activity” or “function” of an enzyme designate the reaction that is catalyzed by said enzyme for converting its corresponding substrate(s) into another molecule(s) (i.e., product(s)). As is well-known in the art, the activity of an enzyme may be assessed by measuring its catalytic efficiency and/or Michaelis constant. Such an assessment is described for example in Segel, 1993, in particular on pages 44-54 and 100-112, incorporated herein by reference.

Preferably, said diaminobutyric acid acetyltransferase has at least 90% similarity with SEQ ID NO: 1, SEQ ID NO: 2, SEQ ID NO: 3, SEQ ID NO: 4 or SEQ ID NO: 5. Preferably, said diaminobutyric acid acetyltransferase is EctA of Halomonas elongata (SEQ ID NO: 1), or a functional fragment, or functional variant thereof.

Preferably, said diaminobutyric acid aminotransferase has at least 90% similarity with SEQ ID NO: 6, SEQ ID NO: 7, SEQ ID NO: 8, SEQ ID NO: 9 or SEQ ID NO: 10. Preferably, said diaminobutyric acid aminotransferase is EctB of H. elongata (SEQ ID NO: 6), or a functional fragment, or functional variant thereof.

Preferably, said ectoine synthase has at least 90% similarity with SEQ ID NO: 11, SEQ ID NO: 12, SEQ ID NO: 13, SEQ ID NO: 14 or SEQ ID NO: 15. Preferably, said ectoine synthase is EctC of H. elongata (SEQ ID NO: 11), or a functional fragment, or functional variant thereof.

A “functional fragment” of an enzyme, as used herein, refers to parts of the amino acid sequence of an enzyme comprising at least all the regions essential for exhibiting the biological activity of said enzyme. These parts of sequences can be of various lengths, provided that the biological activity of the amino acid sequence of the enzyme of reference is retained by said parts. In other words, a functional fragment of an enzyme as provided herein is enzymatically active.

A “functional variant” as used herein refers to a protein that is structurally different from the amino acid sequence of a protein of reference but that generally retain all the essential functional characteristics of said protein of reference. A variant of a protein may be a naturally-occurring variant or a non-naturally occurring variant. Such non-naturally occurring variants of the reference protein can be made, for example, by mutagenesis techniques on the encoding nucleic acids or genes, for example by random mutagenesis or site-directed mutagenesis.

Structural differences may be limited in such a way that the amino acid sequence of reference protein and the amino acid sequence of the variant may be closely similar overall, and identical in many regions. Structural differences may result from conservative or non-conservative amino acid substitutions, deletions and/or additions between the amino acid sequence of the reference protein and the variant. The only proviso is that, even if some amino acids are substituted, deleted and/or added, the biological activity of the amino acid sequence of the reference protein is retained by the variant. As a non-limiting example, such a variant of an ectoine synthase conserves its capacity of transforming Nγ-acetyl-L-2,4-diaminobutyric acid into ectoine. The capacity of the variants to exhibit such activity can be assessed according to in vitro tests known to the person skilled in the art. It should be noted that the activity of said variants may differ in efficiency as compared to the activity of the amino acid sequences of the enzymes of reference provided herein (e.g., the genes/enzymes provided herein of a particular species of micro-organism or having particular sequences as provided in the corresponding SEQ ID NO).

A “functional variant” of an enzyme as described herein includes, but is not limited to, enzymes having amino acid sequences which are at least 60% similar or identical after alignment to the amino acid sequence encoding an enzyme as provided herein. According to the present invention, such a variant preferably has at least 70%, 80%, 85%, 90%, 95%, 96%, 97%, 98%, 99%, or 100% amino acid sequence similarity or identity to the protein described herein. Said functional variant furthermore has the same enzymatic function as the enzyme provided herein. As a non-limiting example, a functional variant of ectoine synthase of SEQ ID NO: 11 has at least 70%, 80%, 85%, 90%, 95%, 96%, 97%, 98%, 99%, or 100% sequence similarity to said sequence. As a non-limiting example, means of determining sequence similarity are further provided below.

Preferably, the expression of at least one of said enzymes, more preferably all three enzymes, is due to an expression of the gene(s) coding for said enzyme(s).

Preferably, said attenuation of pyruvate kinase activity results from an inhibition of expression of the pykA and pykF genes. More preferably, said pykA and pykF genes are deleted.

Thus, said microorganism genetically modified for the production of ectoine preferably comprises the following modifications:
expression of
   heterologous gene ectA encoding a diaminobutyric acid acetyltransferase having at least 90% similarity with SEQ ID NO: 1, SEQ ID NO: 2, SEQ ID NO: 3, SEQ ID NO: 4 or SEQ ID NO: 5,
   heterologous gene ectB encoding a diaminobutyric acid aminotransferase having at least 90% similarity with SEQ ID NO: 6, SEQ ID NO: 7, SEQ ID NO: 8, SEQ ID NO: 9 or SEQ ID NO: 10, and
   heterologous gene ectC encoding an ectoine synthase having at least 90% similarity with SEQ ID NO: 11, SEQ ID NO: 12, SEQ ID NO: 13, SEQ ID NO: 14 or SEQ ID NO: 15,
and
deletion of pykA and pykF genes.

According to a particular embodiment, said microorganism genetically modified for the production of ectoine preferably comprises the following modifications:
expression of
   heterologous gene ectA encoding a diaminobutyric acid acetyltransferase having at least 80% identity, more preferably at least 90% identity, even more preferably at least 95% identity, with SEQ ID NO: 1, 2, 3, 4 or 5,
   heterologous gene ectB encoding a diaminobutyric acid aminotransferase having at least 80% identity, more preferably at least 90% identity, even more preferably at least 95% identity, with SEQ ID NO: 6, 7, 8, 9 or 10, and
   heterologous gene ectC encoding an ectoine synthase having at least 80% identity, more preferably at least 90% identity, even more preferably at least 95% identity, with SEQ ID NO: 11, 12, 13, 14 or 15, and
deletion of pykA and pykF genes.

Said ectA gene preferably encodes the EctA protein having the sequence of SEQ ID NO: 1. Said ectB gene preferably encodes the EctB protein having the sequence of SEQ ID NO: 6. Said ectC gene preferably encodes the EctC protein having the sequence of SEQ ID NO: 11.

Preferably said pykA gene has the sequence of SEQ ID NO: 16. Preferably, said pykF gene has the sequence of SEQ ID NO: 17.

In addition to the modifications described above, the genetically modified microorganism for production of ectoine may comprise one or more additional modifications among those described below.

In particular, said microorganism may further comprise a reduction in citrate synthase activity. Preferably, said microorganism comprises at least a 50% reduction in citrate synthase activity as compared to an unmodified microorganism, more preferably at least a 75% reduction, even more preferably at least a 90% reduction, most preferably at least a 95% reduction, in citrate synthase activity. Preferably, citrate synthase activity is reduced though not completely inhibited. Preferably, said citrate synthase has at least 90% similarity with SEQ ID NO: 18, SEQ ID NO: 19, SEQ ID NO: 20, or SEQ ID NO: 21, said enzyme being encoded by the gltA gene. According to a particular embodiment, said citrate synthase has at least 80% identity, more preferably at least 90% identity, even more preferably at least 95% identity, most preferably 100% identity, with SEQ ID NO: 18, 19, 20, or 21. Preferably, said gltA gene encodes the GltA protein having at least 90% identity with the SEQ ID NO: 18.

Said gltA gene may be under the control of a homologous or heterologous inducible promoter. As a non-limiting example, gltA may be under the control of PgltA (SEQ ID NO: 22) (its wild-type promoter) or under the control of a heterologous inducible promoter, such as Ptrc (SEQ ID NO: 23), Ptac (SEQ ID NO: 24), Plac (SEQ ID NO: 25), Ptet (SEQ ID NO: 26), PL (SEQ ID NO: 27), or PR (SEQ ID NO: 28). Indeed, the use of such a promoter allows gene expression to be controlled (i.e., reduced) according to the presence or absence of a stimuli (e.g., a molecule, temperature, oxygen, or light) which ultimately leads to a reduction in enzyme activity as lower levels of protein are produced. The heterologous inducible promoter may be positively or negatively induced in the presence of a stimuli. Preferably, said gltA gene is under the control PgltA, preferably having the sequence of SEQ ID NO: 21, or a heterologous inducible promoter selected from Ptrc, preferably having the sequence of SEQ ID NO: 22, Ptac, preferably having the sequence of SEQ ID NO: 23, Plac, preferably having the sequence of SEQ ID NO: 24, Ptet, preferably having the sequence of SEQ ID NO: 25, PL, preferably having the sequence of SEQ ID NO: 26, or PR, preferably having the sequence of SEQ ID NO: 27. More preferably, said heterologous inducible promoter is selected from PgltA, Ptet, PL, and PR.

The microorganism genetically modified for production of ectoine may further comprise an attenuation in the activity of a phosphoenolpyruvate carboxylase (also referred to as PEPC or Ppc). The microorganism genetically modified for production of ectoine may further comprise an increase in the activity of a phosphoenolpyruvate carboxykinase (also referred to as PEPCK or Pck). Preferably, activity of the Ppc enzyme is attenuated while activity of the Pck enzyme is increased. Pck may be an enzyme that is endogenous or heterologous to the microorganism. As a non-limiting example, Pck may be from E. coli or Anaerobiospirillum succiniciproducens. Preferably, the amino acid sequence of Pck has at least 90% similarity with the sequence of SEQ ID NO: 29, SEQ ID NO: 30, SEQ ID NO: 31, SEQ ID NO: 32 or of SEQ ID NO: 33.

According to a particular embodiment, the amino acid sequence of Pck has at least 80% identity, more preferably at least 90% identity, even more preferably at least 95% identity, most preferably 100% identity, with the sequence of SEQ ID NO: 29, 30, 31, 32 or 33.

According a particularly preferred embodiment, the ppc gene coding for the PEPC is deleted while the pck gene coding for PEPCK is overexpressed. Even more preferably, the pck gene is introduced into the microorganism at the ppc locus, thereby causing the deletion of ppc. Preferably, said ppc gene has at least 80% sequence identity, more preferably at least 90% identity, even more preferably at least 95% identity, most preferably 100% identity, with the sequence of SEQ ID NO: 34. Preferably, said pck gene encodes the Pck protein having the sequence of SEQ ID NO: 30.

The microorganism genetically modified for production of ectoine may further comprise overexpression of an aspartate transaminase. Preferably, said aspartate transaminase has at least 90% similarity with SEQ ID NO: 35, SEQ ID NO: 36, SEQ ID NO: 37 or SEQ ID NO: 38. According to a particular embodiment, the amino acid sequence of the aspartate transaminase has at least 80% identity, more preferably at least 90% identity, even more preferably at least 95% identity, most preferably 100% identity, with the sequence of SEQ ID NO: 35, 36, 37 or 38. Preferably, said aspartate transaminase is endogenous. Thus, when said microorganism is E. coli, said aspartate transaminase is preferably present in E. coli. Preferably, the aspC gene coding for said aspartate transaminase is overexpressed. Preferably, said aspC gene encodes the AspC protein having the sequence of SEQ ID NO: 35.

The microorganism genetically modified for production of ectoine may further comprise overexpression of a glutamate dehydrogenase. Preferably, said glutamate dehydrogenase has at least 90% similarity with SEQ ID NO: 39, SEQ ID NO: 40, SEQ ID NO: 41, SEQ ID NO: 42, SEQ ID NO: 43, or SEQ ID NO: 44. According to a particular embodiment, the amino acid sequence of the glutamate dehydrogenase has at least 80% identity, more preferably at least 90% identity, even more preferably at least 95% identity, most preferably 100% identity, with the sequence of SEQ ID NO: 39, 40, 41, 42, 43 or 44. Preferably, said glutamate dehydrogenase is heterologous to the microorganism genetically modified for the production of ectoine. Said glutamate dehydrogenase may notably be from Bacillus subtilis. Preferably, said glutamate dehydrogenase has a dependency and/or sensitivity to NADH that is equivalent or superior to that observed for the glutamate dehydrogenase of B. subtilis. Preferably, the gene coding for said glutamate dehydrogenase (e.g., rocG) is overexpressed. Preferably, said rocG gene encodes the RocG protein having the sequence of SEQ ID NO: 39.

Preferably, said microorganism genetically modified for the production of ectoine comprises an overexpression of gene aspC encoding an aspartate transaminase as provided herein and/or gene rocG encoding a glutamate dehydrogenase as provided herein. According to a preferred embodiment, both aspartate transaminase having at least 90% similarity with SEQ ID NO: 35, SEQ ID NO: 36, SEQ ID NO: 37 or SEQ ID NO: 38, and a glutamate dehydrogenase having at least 90% similarity with SEQ ID NO: 39, SEQ ID NO: 40, SEQ ID NO: 41, SEQ ID NO: 42, SEQ ID NO: 43, or SEQ ID NO: 44 are overexpressed. More preferably, said microorganism genetically modified for the production of ectoine comprises an overexpression of gene aspC encoding aspartate transaminase of SEQ ID NO: 35 and gene rocG encoding glutamate dehydrogenase of SEQ ID NO: 39. When both the aspC and rocG genes are overexpressed, a copy of each of said genes may be present on a plasmid. In some cases, both of said genes may be present on the same plasmid.

The microorganism genetically modified for production of ectoine may further comprise a deletion of at least one gene selected from the group consisting of ackA-pta, adhE, frdABCD, IdhA, mgsA, pflAB, and mdh. Said genes are notably endogenous in E. coli. Preferably, said ackA-pta genes have the sequence having at least 80% sequence identity, more preferably at least 90% identity, even more preferably at least 95% identity, most preferably 100% identity, with SEQ ID NO: 45 and SEQ ID NO: 46, respectively. Preferably, said adhE gene has a sequence having at least 80% sequence identity, more preferably at least 90% identity, even more preferably at least 95% identity, most preferably 100% identity, with SEQ ID NO: 47. Preferably, said frdABCD genes have the sequences having at least 80% sequence identity, more preferably at least 90% identity, even more preferably at least 95% identity, most preferably 100% identity, with SEQ ID NO: 48, 49, 50, and 51, respectively. Preferably, said IdhA gene has a sequence having at least 80% sequence identity, more preferably at least 90% identity, even more preferably at least 95% identity, most preferably 100% identity, with SEQ ID NO: 52. Preferably, said mgsA gene has a sequence having at least 80% sequence identity, more preferably at least 90% identity, even more preferably at least 95% identity, most preferably 100% identity, with SEQ ID NO: 53. Preferably, said pflAB genes have the sequences having at least 80% sequence identity, more preferably at least 90% identity, even more preferably at least 95% identity, most preferably 100% identity, with SEQ ID NO: 54 and 55. Preferably, said deletion is a complete deletion of the coding region of each of said genes.

The microorganism genetically modified for the production of ectoine may further comprise a deletion of at least one gene selected from dcuA and aspA. Said genes are notably endogenous in E. coli. Preferably, said dcuA gene has a sequence having at least 80% sequence identity, more preferably at least 90% identity, even more preferably at least 95% identity, most preferably 100% identity, with SEQ ID NO: 56. Preferably, said aspA gene has a sequence having at least 80% sequence identity, more preferably at least 90% identity, even more preferably at least 95% identity, most preferably 100% identity, with SEQ ID NO: 57.

In a further aspect, the microorganism genetically modified for the production of ectoine as described herein is further modified to be able to use sucrose as a carbon source. Preferably, proteins involved in the import and metabolism of sucrose are overexpressed. Preferably, the following proteins are overexpressed:
CscB sucrose permease, CscA sucrose hydrolase, CscK fructokinase, and CscR csc-specific repressor, or
ScrA Enzyme II of the phosphoenolpyruvate-dependent phosphotransferase system and, said ScrK gene encodes ATP-dependent fructokinase, said ScrB sucrose 6-phosphate hydrolase (invertase), said ScrY sucrose porine, ScrR sucrose operon repressor.

Preferably, genes coding for said proteins are overexpressed according to one of the methods provided herein. Preferably, the E. coli microorganism overexpresses:
the heterologous cscBKAR genes of E. coli EC3132, or
the heterologous scrKYABR genes of Salmonella sp.

Genes and proteins are identified herein using the denominations of the corresponding genes in E. coli (e.g., E. coli K12 MG1655 having the Genbank accession number 000096.3) unless otherwise specified. However, in some cases use of these denominations has a more general meaning according to the invention and covers all of the corresponding genes and proteins in microorganisms. This is notably the case for the genes and proteins described herein that are not present in the microorganism (i.e., that are heterologous), such as ectoine synthase, glutamate dehydrogenase, etc. Reference provided herein to any protein (e.g., enzyme) or gene further comprises functional fragments, mutants, and functional variants thereof. As provided herein, said functional fragments, mutants, and functional variants preferably have at least 90% similarity to said protein or gene, or alternatively, at least 80%, 90%, 95%, or even 100%, identity to said protein or gene.

A degree of sequence identity between proteins is a function of the number of identical amino acid residues or nucleotides at positions shared by the sequences of said proteins. The term “sequence identity” or “identity” as used herein in the context of two nucleotide or amino acid sequences more particularly refers to the residues in the two sequences that are the identical when aligned for maximum correspondence. When percentage of sequence identity is used in reference to amino acid sequences, it is recognized that positions at which amino acids are not identical often differ by conservative amino acid substitutions, where amino acid residues are substituted for other amino acid residues having similar chemical properties (e.g., charge or hydrophobicity). When sequences differ due to conservative substitutions, percent identity between sequences may be adjusted upwards to correct for the conservative nature of the substitution. Sequences that differ by such conservative substitutions are said to have “sequence similarity” or “similarity”. Thus, the degree of sequence similarity between polypeptides is a function of the number of similar amino acid residues at positions shared by the sequences of said proteins. The means of identifying similar sequences and their percent similarity or their percent identities are well-known to those skilled in the art, and include in particular the BLAST programs, which can be used from the website http://www.ncbi.nlm.nih.gov/BLAST/ with the default parameters indicated on that website. The sequences obtained can then be exploited (e.g., aligned) using, for example, the programs CLUSTALW (http://www.ebi.ac.uk/clustalw/) or MULTALIN (http://prodes.toulouse.inra.fr/multalin/cgi-bin/multalin.pl), with the default parameters indicated on those websites.

Using the references given in GenBank for known genes, the person skilled in the art is able to determine the equivalent genes in other organisms, bacterial strains, yeasts, fungi, mammals, plants, etc. This routine work is advantageously done using consensus sequences that can be determined by carrying out sequence alignments with genes derived from other microorganisms, and designing degenerate probes to clone the corresponding gene in another organism. These routine methods of molecular biology are well-known to those skilled in the art.

Specifically, sequence similarity and sequence identity between amino acid sequences can be determined by comparing a position in each of the sequences which may be aligned for the purposes of comparison. When a position in the compared sequences is occupied by a similar amino acid or by the same amino acid then the sequences are, respectively, similar or identical at that position.

Sequence similarity may notably be expressed as the percent similarity of a given amino acid sequence to that of another amino acid sequence. This refers to the similarity between sequences on the basis of a “similarity score” that is obtained using a particular amino acid substitution matrix. Such matrices and their use in quantifying the similarity between two sequences are well-known in the art and described, for example in Dayhoff et al., 1978, and in Henikoff and Henikoff, 1992. Sequence similarity may be calculated from the alignment of two sequences, and is based on a substitution score matrix and a gap penalty function. As a non-limiting example, the similarity score is determined using the BLOSUM62 matrix, a gap existence penalty of 10, and a gap extension penalty of 0.1 or the BLOSUM62 matrix, a gap existence penalty of 11, and a gap extension penalty of 1. Preferably, no compositional adjustments are made to compensate for the amino acid compositions of the sequences being compared and no filters or masks (e.g., to mask off segments of the sequence having low compositional complexity) are applied when determining sequence similarity using web-based programs, such as BLAST. The maximum similarity score obtainable for a given amino acid sequence is that obtained when comparing a sequence with itself. For example, the maximum similarity score obtainable for SEQ ID NO:1 is 1015 using the above described parameters (i.e., using the BLOSUM62 matrix, a gap existence penalty of 10, and a gap extension penalty of 0.1 or alternatively a gap existence penalty of 11, and a gap extension penalty of 1). As a further example, the maximum score is 1008 for SEQ ID NO: 2, 1016 for SEQ ID NO: 3, 993 for SEQ ID NO: 4, 891 for SEQ ID NO: 5, 2192 for SEQ ID NO: 6, 2214 for SEQ ID NO: 7, 2221 for SEQ ID NO: 8, 2215 for SEQ ID NO: 9, 2225 for SEQ ID NO: 10, 744 for SEQ ID NO: 11, 714 for SEQ ID NO: 12, 707 for SEQ ID NO: 13, 696 for SEQ ID NO: 14, and 695 for SEQ ID NO: 15. The skilled person is able to determine such maximum similarity scores on the basis of the above-described parameters for any amino acid sequence. A statistically relevant similarity can furthermore be indicated by a “bit score” as described, for example, in Durbin et al., Biological Sequence Analysis, Cambridge University Press (1998).

To determine if a given amino acid sequences has at least 90% similarity with a protein provided herein, said amino acid sequence can be optimally aligned as provided above, preferably using the BLOSUM62 matrix, a gap existence penalty of 10, and a gap extension penalty of 0.1. Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. A sequence having 90% similarity with such SEQ ID NO: 1 using the above-described parameters will have a score of at least 914. 90% similarity corresponds to a score of at least 908 for SEQ ID NO: 2, at least 915 for SEQ ID NO: 3, 894 for SEQ ID NO: 4, 802 for SEQ ID NO: 5, 1973 for SEQ ID NO: 6, 1993 for SEQ ID NO: 7, 1999 for SEQ ID NO: 8, 1994 for SEQ ID NO: 9, 2003 for SEQ ID NO: 10, 670 for SEQ ID NO: 11, 643 for SEQ ID NO: 12, 634 for SEQ ID NO: 13, 627 for SEQ ID NO: 14, and 626 for SEQ ID NO: 15. The skilled person is able to determine 90% similarity with a maximum score determined on the basis of the above-described parameters for any amino acid sequence.

Percent similarity or percent identities as referred to herein are determined after optimal alignment of the sequences to be compared, which may therefore comprise one or more insertions, deletions, truncations and/or substitutions. This percent identity may be calculated by any sequence analysis method well-known to the person skilled in the art. The percent similarity or percent identity may be determined after global alignment of the sequences to be compared of the sequences taken in their entirety over their entire length. In addition to manual comparison, it is possible to determine global alignment using the algorithm of Needleman and Wunsch (1970). Optimal alignment of sequences may preferably be conducted by the global alignment algorithm of Needleman and Wunsch (1970), by computerized implementations of this algorithm (such as CLUSTAL W) or by visual inspection.

For nucleotide sequences, the sequence comparison may be performed using any software well-known to a person skilled in the art, such as the Needle software. The parameters used may notably be the following: “Gap open” equal to 10.0, “Gap extend” equal to 0.5, and the EDNAFULL matrix (NCBI EMBOSS Version NUC4.4).

For amino acid sequences, the sequence comparison may be performed using any software well-known to a person skilled in the art, such as the Needle software. The parameters used may notably be the following: “Gap open” equal to 10, “Gap extend” equal to 0.1, and the BLOSUM62 matrix.

Preferably, the percent similarity or identity as defined herein is determined via the global alignment of sequences compared over their entire length.

As a particular example, to determine the percentage of similarity or identity between two amino acid sequences, the sequences are aligned for optimal comparison. For example, gaps can be introduced in the sequence of a first amino acid sequence for optimal alignment with the second amino acid sequence. The amino acid residues at corresponding amino acid positions are then compared. When a position in the first sequence is occupied by a different but conserved amino acid residue, the molecules are similar at that position, and accorded a particular score (e.g., as provided in a given amino acid substitution matrix, discussed previously). When a position in the first sequence is occupied by the same amino acid residue as the corresponding position in the second sequence, the molecules are identical at that position.

The percentage of identity between the two sequences is a function of the number of identical positions shared by the sequences. Hence % identity=number of identical positions/total number of overlapping positions×100.

In other words, the percentage of sequence identity is calculated by comparing two optimally aligned sequences, determining the number of positions at which the identical amino acid occurs in both sequences to yield the number of matched positions, dividing the number of matched positions by the total number of positions and multiplying the result by 100 to yield the percentage of sequence identity.

PFAM (protein family database of alignments and hidden Markov models; http://www.sanger.ac.uk/Software/Pfam/) represents a large collection of protein sequence alignments which may also be consulted by the skilled person. Each PFAM makes it possible to visualize multiple alignments, see protein domains, evaluate distribution among organisms, gain access to other databases, and visualize known protein structures.

Finally, COGs (clusters of orthologous groups of proteins; http://www.ncbi.nlm.nih.gov/COG/) may be obtained by comparing protein sequences from 43 fully sequenced genomes representing 30 major phylogenic lines. Each COG is defined from at least three lines, which permits the identification of former conserved domains.

The above definitions and preferred embodiments related to the functional fragments and functional variants of proteins apply mutatis mutandis to nucleotide sequences, such as genes, encoding said proteins (i.e., a gene encoding an ectoine synthase).

Methods for the Production of Ectoine

According to a further aspect, the present invention relates to a method for the production of ectoine using the microorganism described herein. Said method comprises the steps of:
a) culturing the microorganism genetically modified for the production of ectoine as described herein on an appropriate culture medium comprising a source of carbon and a source of nitrogen, and
b) recovering ectoine from the culture medium.

More specifically, the invention relates to a method for the fermentative production of ectoine using the microorganism described herein. According to the invention, the terms “fermentative process,” “fermentative production,” “fermentation,” or “culture” are used interchangeably to denote the growth of microorganism. This growth is generally conducted in fermenters with an appropriate growth medium adapted to the microorganism being used.

An “appropriate culture medium” designates a medium (e.g., a sterile, liquid media) comprising nutrients essential or beneficial to the maintenance and/or growth of the cell such as carbon sources or carbon substrates, nitrogen sources; phosphorus sources, for example, monopotassium phosphate or dipotassium phosphate; trace elements (e.g., metal salts), for example magnesium salts, cobalt salts and/or manganese salts; as well as growth factors such as amino acids and vitamins. In particular, the inorganic culture medium can be of identical or similar composition to an M9 medium (Anderson, 1946), an M63 medium (Miller, 1992) or a medium such as defined by Schaefer et al. (1999).

The term “source of carbon,” “carbon source,” or “carbon substrate” according to the present invention refers to any carbon source capable of being metabolized by a microorganism wherein the substrate contains at least one carbon atom. According to the present invention, said source of carbon is preferably at least one carbohydrate, and in some cases a mixture of at least two carbohydrates.

The term “carbohydrate” refers to any carbon source capable of being metabolized by a microorganism and containing at least three carbon atoms, two atoms of hydrogen. The one or more carbohydrates may be selected from among the group consisting of: monosaccharides such as glucose, fructose, mannose, xylose, arabinose, galactose and the like, disaccharides such as sucrose, cellobiose, maltose, lactose and the like, oligosaccharides such as raffinose, stacchyose, maltodextrins and the like, polysaccharides such as cellulose, hemicellulose, starch and the like, and glycerol. Especially preferred carbon sources are glycerol, arabinose, fructose, galactose, glucose, lactose, maltose, sucrose, xylose or a mixture of two or more thereof. Most preferably, the carbohydrate is glycerol and/or glucose and/or sucrose.

The term “source of nitrogen” according to the present invention refers to any nitrogen source capable of being used by the microorganism. Said source of nitrogen may be inorganic (e.g., (NH₄)₂SO₄) or organic (e.g., urea or glutamate). Preferably, said source of nitrogen is in the form of ammonium or ammoniac. Preferably, said source of nitrogen is either an ammonium salt, such as ammonium sulfate, ammonium chloride, ammonium nitrate, ammonium hydroxide and ammonium phosphate, or to ammoniac gas, corn steep liquor, peptone (e.g., Bacto™ peptone), yeast extract, meat extract, malt extract, or urea, or any combination thereof. In some cases, the nitrogen source may be derived from renewable biomass of microbial origin (such as beer yeast autolysate, waste yeast autolysate, baker's yeast, hydrolyzed waste cells, algae biomass), vegetal origin (such as cotton seed meal, soy peptone, soybean peptide, soy flour, soybean flour, soy molasses, rapeseed meal, peanut meal, wheat bran hydro lysate, rice bran and defatted rice bran, malt sprout, red lentil flour, black gram, bengal gram, green gram, bean flour, flour of pigeon pea, protamylasse) or animal origin (such as fish waste hydrolysate, fish protein hydrolysate, chicken feather; feather hydrolysate, meat and bone meal, silk worm larvae, silk fibroin powder, shrimp wastes, beef extract), or any other nitrogen containing waste. More preferably, said source of nitrogen is peptone and/or yeast extract.

The term “recovering” as used herein designates the process of separating or isolating the produced ectoine by using conventional laboratory techniques known to the person skilled in the art. Ectoine may by recovered from the culture media and/or from the microorganism itself. Preferably, ectoine is recovered from at least the culture media.

The person skilled in the art is able to define the culture conditions for the microorganisms according to the invention. In particular the bacteria are fermented at a temperature between 20° C. and 55° C., preferably between 25° C. and 40° C., more preferably between about 30° C. to 39° C., even more preferably about 37° C. In cases, where a thermo-inducible promoter is comprised in the microorganism provided herein, said microorganism is preferably fermented at about 39° C.

This process can be carried out either in a batch process, in a fed-batch process or in a continuous process. It can be carried out under aerobic, micro-aerobic or anaerobic conditions, or a combination thereof (for example, aerobic conditions followed by anaerobic conditions).

“Under aerobic conditions” means that oxygen is provided to the culture by dissolving the gas into the liquid phase. This could be obtained by (1) sparging oxygen containing gas (e.g., air) into the liquid phase or (2) shaking the vessel containing the culture medium in order to transfer the oxygen contained in the head space into the liquid phase. The main advantage of the fermentation under aerobic conditions is that the presence of oxygen as an electron acceptor improves the capacity of the strain to produce more energy under the form of ATP for cellular processes. Therefore, the strain has its general metabolism improved.

Micro-aerobic conditions are defined as culture conditions wherein low percentages of oxygen (e.g., using a mixture of gas containing between 0.1 and 15% of oxygen, completed to 100% with inert gas such as nitrogen, helium or argon, etc.), is dissolved into the liquid phase.

Anaerobic conditions are defined as culture conditions wherein no oxygen is provided to the culture medium. Strictly anaerobic conditions are obtained by sparging an inert gas like nitrogen into the culture medium to remove traces of other gas. Nitrate can be used as an electron acceptor to improve ATP production by the strain and improve its metabolism.

Ectoine production, for example in the culture medium, can be determined by standard analytical means known by the person skilled in the art. As a non-limiting example, ectoine may be quantified using HPLC or nuclear magnetic resonance, for example as provided in Kuhlmann and Bremer, 2002, Chen et al., 2017, and Rui-Feng et al., 2017. The sample comprising ectoine may be prepared using ethanol extraction.

Step b) of the method described herein preferably comprises a step of filtration, desalination, cation exchange, liquid extraction, crystallization, or distillation, or combinations thereof. Ectoine may be recovered from both culture medium and microorganisms, or from only one or the other. Preferably, ectoine is recovered from at least the culture medium. The volume of culture medium may be reduced for example via ceramic membrane filtration. Ectoine may furthermore be recovered either during culturing of the microorganism by in situ product recovery including extractive fermentation, or after fermentation is finished. Microorganisms may notably be removed by passing through a device, preferably through a filter with a cut-off in the range from 5 to 200 kDa, where solid/liquid separation takes place. It is also feasible to employ a centrifuge, a suitable sedimentation device or a combination of these devices, it being especially preferred to first separate at least part of the microorganisms by sedimentation and subsequently to feed the fermentation broth, which has been partly relieved of the microorganisms, to ultrafiltration or to a centrifugation device. After the microorganisms have been removed, ectoine present in the remaining culture medium may be recovered. Ectoine may be recovered from microorganisms separately.

Recovery of ectoine from microorganism may involve lysis or disruption by heating to induce ectoine release from microorganisms.

Ectoine may be further purified by using conventional laboratory techniques known to the skilled person, such as filtration and/or crystallization, for example after dissolving ectoine in methanol (Chen et al., 2017).

### FIGURES

FIG. 1: Map of the recombinant vector pEC1.

p15A: plasmid origin of replication; aadA1: aminoglycoside 3′-adenylyltransferase conferring spectinomycin/streptomycin resistance; Placl Q: laclQ promoter; laclQ: mutant lac repressor gene binding the lac operator more tightly than the wild-type Lacl; Ptrc01: artificial promoter with lac operator binding site (Brosius et al., 1985); operateur lac: lac operator binding site; ectA-ectB-ectC: H. elongata ectoine operon.

FIG. 2: Map of the recombinant vector pEC3.

p15A: plasmid origin of replication; aadA1: aminoglycoside 3′-adenylyltransferase conferring spectinomycin/streptomycin resistance; Placl Q: laclQ promoter; laclQ: mutant lac repressor gene binding the lac operator more tightly than the wild-type Lacl; Ptrc01: artificial promoter with lac operator binding site (Brosius et al., 1985); operateur lac: lac operator binding site; ectA-ectB-ectC: H. elongata ectoine operon; rocG: B. subtilis glutamate dehydrogenase gene; PaspC: aspC promoter; aspC: E. coli aspartate transaminase gene

FIG. 3: Metabolic pathway for the production of ectoine from glucose. The first step (1) is the phosphotransferase system (PTS) responsible for both glucose uptake and phosphorylation on the carbon 6. In this schematic representation G6P, PEP, and OAA mean respectively glucose-6-phosphate, phosphoenolpyruvate and oxaloacetic acid. Enzymatic activities are (2) phosphoenolpyruvate carboxykinase (EC: 4.1.1.49), (3) pyruvate decarboxylase (EC: 1.2.4.1), (4) aspartate aminotransferase (EC: 2.6.1.1), (5) glutamate dehydrogenase (EC: 1.4.1.2), (6) aspartokinase (EC: 2.7.2.4), (7) aspartate-semialdehyde dehydrogenase (EC: 1.2.1.11), (8) diaminobutyrate-2-oxoglutarate transaminase (EC: 2.6.1.76), (9) L-2,4-diaminobutyric acid acetyltransferase (EC: 2.3.1.178), (10) L-ectoine synthase (EC: 4.2.1.108). Citrate synthase activity (11) (EC: 2.3.3.1) is strongly regulated by environmental conditions (12).

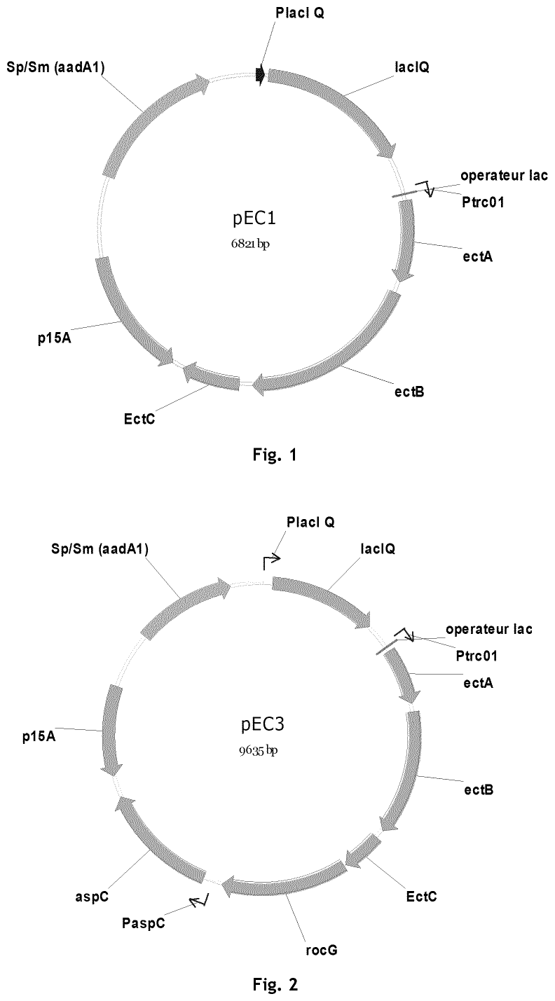

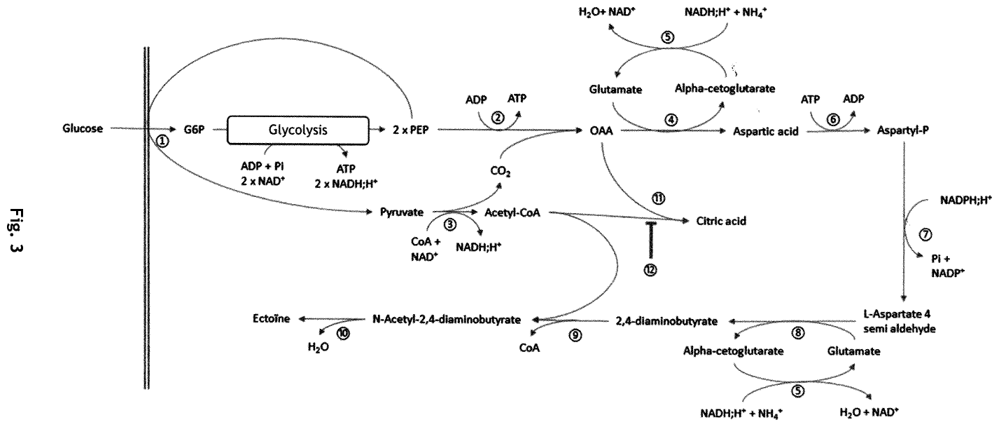

### EXAMPLES

The present invention is further defined in the following examples. It should be understood that these examples, while indicating preferred embodiments of the invention, are given by way of illustration only. The person skilled in the art will readily understand that these examples are not limitative and that various modifications, substitutions, omissions, and changes may be made without departing from the scope of the invention.

### Methods

In the examples given below, methods well-known in the art were used to construct E. coli strains containing replicating vectors and/or various chromosomal deletions, and substitutions using homologous recombination, as is well-described in Datsenko & Wanner, (2000) for E. coli. In the same manner, the use of plasmids or vectors to express or overexpress one or more genes in a recombinant microorganism are well-known by the person skilled in the art. Examples of suitable E. coli expression vectors include pTrc, pACYC184, pBR322, pUC18, pUC19, pKC30, pRep4, pHS1, pHS2, pPLc236, etc.

Chromosomal modifications. Several protocols have been used in the following examples. Protocol 1 (chromosomal modifications by PCR amplification using oligonucleotides and appropriate genomic DNA as a matrix (that the person skilled in the art will be able to define), homologous recombination and selection of recombinants), protocol 2 (transduction of phage P1) and protocol 3 (antibiotic cassette excision, the resistance genes were removed when necessary) used in this invention have been fully described in patent application EP 2532751 (see in particular example 1 and example 3, points 1.2 and 1.3, incorporated herein by reference). Chromosomal modifications were verified by PCR analysis with appropriate oligonucleotides that the person skilled in the art is able to design.

Construction of recombinant plasmids. Recombinant DNA technology is well described in the literature and routinely used by the person skilled in the art. Briefly, DNA fragments were PCR amplified using oligonucleotides and appropriate genomic DNA as a matrix (that the person skilled in the art will be able to define). The DNA fragments and chosen plasmid were digested with compatible restriction enzymes (that the person skilled in the art will be able to define), then ligated and transformed into competent cells. Transformants were analyzed and recombinant plasmids of interest were verified by DNA sequencing.

Overlap extension PCR method (OE-PCR). The overlap extension PCR method consists of two or more primary PCR reactions which generate DNA fragments with overlapping ends and a secondary reaction which joins the two or more fragments into a single fragment (Horton et al., 1990). The person skilled in the art will be able to define appropriate oligonucleotides for this purpose.

Strain selection based on the expression of genes responsible for antibiotic resistance. Strains construction requires the selection of cells harboring a DNA fragment responsible for a specific antibiotic resistance. To achieve this selection, bacteria are spread on petri dishes containing LB solid medium (10 g/L bactopeptone, 5 g/L yeast extract, 5 g/L NaCl and 20 g/L agar). Three antibiotics could be added according to the selection marker:
Chloramphenicol (30 mg/L)
Kanamycin (50 mg/L)
Gentamycin (10 mg/L)
Spectinomycin (50 mg/L)
Streptomycin (100 mg/L)

Determination of ectoine and acetic acid production. Ectoine and acetic acid concentrations are determined using an ultra high-pressure liquid chromatography system (UPLC ACQUITY, Waters®). Samples collected at different time points are centrifuged for 2 minutes at 5,000 g and at 4° C. to remove the insoluble part. The upper phase of each sample is diluted 1000-fold in distilled water. Ectoine and acetic acid are then separated on an Acquity UPLC-HSS-T3-C18/2.1 mm×150 mm×1.8 μm column (Waters®) using a water/acetonitrile gradient as the mobile phase. The gradient table is presented in Table 1 below.

TABLE 1
Gradient table for acetic acid and ectoine separation.
The total flow rate is constant at 400 μL/minute.

| Time course - | Percent of Solvant A | Percent of Solvant B |
|---|---|---|
| min | (water/acetonitrile 99/1 v/v) | (acetonitrile) |
| 0 | 100 | 0 |
| 4.5 | 100 | 0 |
| 5.5 | 50 | 50 |
| 6 | 10 | 90 |
| 8 | 10 | 90 |
| 9 | 100 | 0 |
| 11 | 100 | 0 |

Ectoine and acetic acid are quantified using the mass spectrometer API3200 (Sciex®).

Biomass estimation. Biomass quantity variation is monitored using a spectrophotometer (Nicolet Evolution 100 UV-Vis, THERMO®). Biomass production increases the turbidity of the culture medium. It is assayed by measuring the absorbance at 600 nm. Each unit of absorbance represents 2.2×10⁹+/−2×10⁸ cells/mL.

Determination of citrate synthase activity. Citrate synthase activity is defined as the ability of a protein mixture to condense the acetyl group from acetyl-CoA and an oxaloacetic Acid (OAA) into citric acid. Such a reaction also releases a co-enzyme A (CoA) harboring a free thiol group. The principle of the present assay is to monitor the release of the free thiol group. To do this, the method described by Georges Ellman in 1959 (Ellman GL, 1959) was adapted. Ellman's reagent (5,5′-dithiobis-(2-nitrobenzoic acid)), also called DTNB, which reacts with free thiol groups, was used. This reagent cleaves the disulfide bond to release 2-nitro-5-thiobenzoate (TNB⁻), which ionizes to the TNB²⁻ dianion in water at neutral and alkaline pH. This TNB²⁻ ion has a yellow color. This reaction is stoichiometric and the molar extinction coefficient of TNB²⁻ at 412 nm is 13,600 mol·L⁻¹·cm⁻¹. A bacterial crude extract is prepared through mechanical grinding (i.e., using glass beads or a french press) or bacterial cell membrane permeabilization (i.e., using guanidine-HCl and Triton X100 treatment). Proteins of interest are separated from cell debris by centrifugation (1,500 g at 4° C.).

The initial assay mix contained from 2 to 6 μg/mL of protein extract, 20 mM HEPES (pH=7.5), 0.15 mM Acetyl-CoA, 0.2 mM DTNB, 0.3 mM OAA. The apparition of the yellow color as a function of time is linearly correlated with specific enzymatic activity and is monitored by analyzing the absorbance at 420 nm with a spectrophotometer (Nicolet Evolution 100 UV-Vis, THERMO®).

### Example 1: Strain Construction

To overexpress the ectoine operon, the ectA, ectB and ectC genes from Halomonas elongata (SEQ ID NO: 1) were cloned under the IPTG inducible trc promoter of SEQ ID NO: 58 into the pACYC184 plasmid (Chang and Cohen, 1978).

The resulting plasmid pEC0001 was then transformed into strain MG1655 giving rise to strain 1.

To optimize the strain for ectoine production, combinations of mutations were introduced into the E. coli strain resulting in strains 2a: MG1655 DackA+pta DadhE DldhA DfrdABCD DmgsA DpflAB Dmdh DaspA DdcuA and 2b: strain 2a with DpykA DpykF, constructed as described below.

To inactivate the ackA+pta, frdABCD, pflAB operons and the adhE, IdhA, mgsA, mdh, aspA, dcuA, pykA, and pykF genes, the homologous recombination strategy was used (according to Protocol 1). The strains retained were designated MG1655 DackA+pta::Gt, MG1655 DadhE::Cm, MG1655 DldhA::Km, MG1655 DfrdABCD::Gt, MG1655 DmgsA::Km, MG1655 DpflAB::Cm, MG1655 Dmdh::Gt, MG1655 DaspA::Km, MG1655 DdcuA::Cm, MG1655 DpykA::Cm and MG1655 DpykF::Km were Km, Cm and Gt designate respectively DNA sequence conferring resistance to kanamycin, chloramphenicol and gentamycin. All these deletions were transferred by P1 phage transduction (according to Protocol 2) into E. coli MG1655 and resistance genes were removed according to protocol 3 when necessary. The resulting strains were named strain 2a and 2b, as indicated above.

The aspC gene from E. coli and the glutamate dehydrogenase gene rocG from B. subtilis (SEQ ID NO: 59) were cloned under the native promoter and the inducible trc promoter SEQ ID NO: 23, respectively, into the previously described pEC0001 plasmid. The resulting plasmid pEC0003 was then transformed into strains 2a and 2b giving rise respectively to strains 3a and 3b.

To increase ectoine production, the phosphoenolpyruvate carboxykinase pck gene from A. succiniciproducens (SEQ ID NO: 60) was chromosomally overexpressed under the inducible trc promoter into the ppc locus, thereby deleting the ppc gene. Specifically, a fragment carrying the native pck gene and a resistance marker flanked by homologous DNA sequences to the targeted integration locus ppc was PCR amplified by OE-PCR. The PCR product obtained was then introduced by electroporation into E. coli strain MG1655 (pKD46). pck overexpression was transferred by P1 phage transduction (according to Protocol 2) into strain 3b and the resistance gene was removed according to protocol 3, giving rise to strain 4.

Results

As can be seen in Table 2, strains 1, 3a, 3b and 4 showed an increase in ectoine production. Ectoine production was better in strain 3b as compared to strain 3a (data not shown), therefore strain 3b was used in the examples below.

TABLE 2
Ectoine production, in batch cultures for the different strains.

| | Ectoine production |
|---|---|
| Control Strain | − |
| Strain 1 | + |
| Strain 3a | + |
| Strain 3b | + |
| Strain 4 | + |

The symbol “+” indicates detectable ectoine in the medium. The “−” represents undetectable ectoine.

### Example 2: CitS Reduction

Biological regulation of citrate synthase activity. In all cases, the sequence of the citrate synthase (gltA) open reading frame (SEQ ID NO: 61) is that present in the wild-type strain (MG1655) or an equivalent nucleotide sequence coding a protein with at least 90% sequence similarity based on Blosum62 homology matrix as compared to native E. coli citrate synthase.

The attenuation of expression can notably be due to either the exchange of the wild-type promoter for a weaker natural or synthetic promoter or the use of an agent reducing gene expression, such as an antisense RNA or interfering RNA (RNAi), and more particularly a small interfering RNA (siRNA) or short hairpin RNA (shRNA).

Regulation systems are controlled by environmental conditions responsible for repression of citrate synthase gene expression. Environmental conditions are listed in Table 3 below. The growth medium “nitrogen base” is composed of 10 g/L bactopeptone and 5 g/L yeast extract supplemented with 0.5 g/L sodium chloride.

TABLE 3
List of environmental culture conditions used.

| Medium name | 1 | 2 | 3 | 4 | 5 |
|---|---|---|---|---|---|
| Nitrogen Base | 1x | 1x | 1x | 1x | 1x |
| Glucose (6 g/L) | + | + | + | + | + |
| Temperature (in ° C.) | 37 | 37 | 30 | 37 | 37 |
| Oxygen pO₂ en mm Hg | 160 | 45 | 160 | 160 | 160 |
| IPTG 100 μM | − | − | − | + | − |
| Anhydrotetracycline 0.4 μM | − | − | − | − | + |

We also used strains in which the genomic sequence of the citrate synthase gene is under the control of a different promoter associated with a specific transcriptional factor, if needed. Strain names and regulator systems used are listed in Table 4 below.

TABLE 4
List of strains used for analysis of citrate synthase regulation.

| Strain | A | B | C | D | E | F | G |
|---|---|---|---|---|---|---|---|
| Regulator | PgltA | Ptrc | Ptac | Plac | Ptet | PL | PR |
| controlling | | | | | | | |
| gltA | | | | | | | |
| SEQ ID | SEQ | SEQ | SEQ | SEQ | SEQ | SEQ | SEQ |
| NO: | ID | ID | ID | ID | ID | ID | ID |
| | NO: | NO: | NO: | NO: | NO: | NO: | NO: |
| | 22 | 23 | 24 | 25 | 26 | 27 | 28 |

Initial biomass is obtained after an overnight culture (16 hours) in condition 4 (see Table 3). All bacteria present in said biomass are in physiological stationary phase due to carbon starvation. The biomass is diluted 100-fold in new medium. After 4 hours in the indicated growth conditions, citrate synthase activity is monitored as previously described. Table 5 provides the obtained results.

TABLE 5
Citrate synthase activity. The “+” indicates a strain/condition
association leading to an activity between 0.8 and 1.2 fold
of the reference measured in strain A and condition 1. The represents
a strain/condition association with an activity below 0.8-fold
of the reference. The “++” means that citrate
synthase activity is increased by more than 1.2-fold of the
reference when the said strain is grown in the indicated condition.

| | | Strains | | | | | | |
|---|---|---|---|---|---|---|---|---|
| | | A | B | C | D | E | F | G |
| Culture | 1 | reference | − | − | − | + | + | + |
| conditions | 2 | − | − | − | − | + | + | + |
| | 3 | + | − | − | − | + | − | − |
| | 4 | + | ++ | ++ | + | + | + | + |
| | 5 | + | − | − | − | − | + | + |

Results presented in Table 5 shown that citrate synthase activity is strongly reduced when PL λ or PR λ controls its expression in association with environmental conditions in which the growing temperature is 30° C. Similar results can be observed when pTet controls citrate synthase gene expression and in presence of tetracycline in the medium. When the gltA gene (coding the citrate synthase) is under the control of its native promoter, citrate synthase activity is also strongly reduced if bacteria are growing in oxygen starvation.

For the pTrc, pTac and pLac regulation systems, expression of the citrate synthase is reduced when IPTG is absent from the medium. Such results indicate that the absence of a specific molecule is controlling the gltA expression. In the present context, the PgltA, Ptet, PL and PR systems are particularly preferred for limiting the citrate synthase activity. Strains constructed using these systems are discussed in further detail below.

### Example 3: Optimisation of Carbon Utilisation for Ectoine Production

Strains constructed for ectoine production test. Regulation systems A, E, F and G described in example 2 have been introduced in strains 1, 3b and 4 presented in the example 1 above. These new strains (Table 6) exhibit the same regulation of the citrate synthase activity.

TABLE 6
List of strains used for analysis of ectoine production.

| Genetic Background | Strain 1 | | | |
|---|---|---|---|---|
| Strain | H | I | J | K |
| Regulator controlling gltA | PgltA | Ptet | PL | PR |
| Seq ID | SEQ ID | SEQ ID | SEQ ID | SEQ ID |
| | NO: 22 | NO: 26 | NO: 27 | NO: 28 |
| Genetic Background | Strain 3b | | | |
| Strain | L | M | N | O |
| Regulator controlling gltA | PgltA | Ptet | PL | PR |
| Seq ID | SEQ ID | SEQ ID | SEQ ID | SEQ ID |
| | NO: 22 | NO: 26 | NO: 27 | NO: 28 |
| Genetic Background | Strain 4 | | | |
| Strain | P | Q | R | S |
| Regulator controlling gltA | PgltA | Ptet | PL | PR |
| Seq ID | SEQ ID | SEQ ID | SEQ ID | SEQ ID |
| | NO: 22 | NO: 26 | NO: 27 | NO: 28 |

The production conditions tested are those presented in example 2 above. In all conditions, the medium is supplemented with 100 μM IPTG to induce ectA/B/C gene expression. Results obtained are provided in Table 7 below.

TABLE 7
Ectoine, acetic acid and biomass production.

| | Conditions | | | |
|---|---|---|---|---|
| Strains | 1 | 2 | 3 | 5 |
| H | Ectoine: reference | Ectoine: + | Ectoine: + | Ectoine: + |
| | Acetic acid: reference | Acetic acid: ++ | Acetic acid: + | Acetic acid: + |
| | Biomass: reference | Biomass: − − | Biomass: + | Biomass: + |
| I | Ectoine: + | Ectoine: + | Ectoine: + | Ectoine: + |
| | Acetic acid: + | Acetic acid: + | Acetic acid: + | Acetic acid: ++ |
| | Biomass: + | Biomass: − | Biomass: + | Biomass: − − |
| J | Ectoine: + | Ectoine: + | Ectoine: + | Ectoine: + |
| | Acetic acid: + | Acetic acid: + | Acetic acid: ++ | Acetic acid: + |
| | Biomass: + | Biomass: − | Biomass: − − | Biomass: + |
| K | Ectoine: + | Ectoine: + | Ectoine: + | Ectoine: + |
| | Acetic acid: + | Acetic acid: + | Acetic acid: ++ | Acetic acid: + |
| | Biomass: + | Biomass: − | Biomass: − − | Biomass: + |
| L | Ectoine: + | Ectoine: +++ | Ectoine: + | Ectoine: + |
| | Acetic acid: − | Acetic acid: − | Acetic acid: − | Acetic acid: − |
| | Biomass: + | Biomass: − − | Biomass: + | Biomass: + |
| M | Ectoine: + | Ectoine: + | Ectoine: + | Ectoine: +++ |
| | Acetic acid: − | Acetic acid: − | Acetic acid: − | Acetic acid: − |
| | Biomass: + | Biomass: − | Biomass: + | Biomass: − − |
| N | Ectoine: + | Ectoine: + | Ectoine: +++ | Ectoine: + |
| | Acetic acid: − | Acetic acid: − | Acetic acid: − | Acetic acid: − |
| | Biomass: + | Biomass: − | Biomass: − − | Biomass: + |
| O | Ectoine: + | Ectoine: + | Ectoine: +++ | Ectoine: + |
| | Acetic acid: − | Acetic acid: − | Acetic acid: − | Acetic acid: − |
| | Biomass: + | Biomass: − | Biomass: − − | Biomass: + |
| P | Ectoine: + | Ectoine: +++ | Ectoine: + | Ectoine: + |
| | Acetic acid: − | Acetic acid: − | Acetic acid: − | Acetic acid: − |
| | Biomass: + | Biomass: − − | Biomass: + | Biomass: + |
| Q | Ectoine: + | Ectoine: + | Ectoine: + | Ectoine: +++ |
| | Acetic acid: − | Acetic acid: − | Acetic acid: − | Acetic acid: − |
| | Biomass: + | Biomass: − | Biomass: + | Biomass: − − |
| R | Ectoine: + | Ectoine: + | Ectoine: +++ | Ectoine: + |
| | Acetic acid: − | Acetic acid: − | Acetic acid: − | Acetic acid: − |
| | Biomass: + | Biomass: − | Biomass: − − | Biomass: + |
| S | Ectoine: + | Ectoine: + | Ectoine: +++ | Ectoine: + |
| | Acetic acid: − | Acetic acid: − | Acetic acid: − | Acetic acid: − |
| | Biomass: + | Biomass: − | Biomass: − − | Biomass: + |

The “+” indicates a strain/condition association leading to a production level between 0.8 and 1.2 fold of the reference measured in strain H and condition 1. The “−” indicates a strain/condition association leading to a production level between 0.6 and 0.8 fold of the reference. The “− −” indicates a strain/condition association leading to a production level below 0.6 fold of the reference. The “++” indicates a strain/condition association leading to a production level between 1.2 and 2 fold of the reference. The “+++” means that the production level is equal or superior to 2-fold of the reference when said strain is grown in the indicated condition.

As illustrated in Table 7, the deletion of pykA and pykF in particular, coupled with a reduction of citrate synthase activity (here by limiting gltA gene expression), surprisingly and advantageously improves ectoine production while also reducing acetic acid production in all conditions (compare e.g., results obtained for strains L to O with strains H to K). provides a relatively simple and robust microorganism that may be used to cost-effectively produce ectoine on an industrial scale.

Results presented in Table 7 also show that while a reduction of citrate synthase activity alone advantageously reduce the conversion of the carbon source into biomass, this is not sufficient to improve ectoine production (see strain H/condition 2; strain I/condition 5; strains J and K/condition 3). Indeed, although incorporation of a carbon source into biomass is a limiting factor for ectoine production, a significant part of the acetyl-CoA present in the microorganism is used for acetic acid production due to a disequilibrium between the oxaloacetic acid and acetyl-CoA production.

However, when weak citrate synthase activity is associated with an optimization of the metabolic pathway as described herein, ectoine production is advantageously improved (see Table 7, strain L or strain P/condition 2; strain M or strain Q/condition 5; strains N and O or strain R and S/condition 3).

### REFERENCES

Altschul et al., (1990), J. Mol. Biol, 215 (3): 403-410.
Anderson, (1946), Proc. Natl. Acad. Sci. USA., 32:120-128.
Bantscheff et al., (2007), Analytical and Bioanalytical Chemistry, vol. 389(4): 1017-1031.
Becker et al., 2013, Microbial Cell Factories. 12(1):110
Burnette (1981). Analytical Biochemistry, 112(2): 195-203.
Chang and Cohen, (1978), Journal of Bacteriology, Vol 134 No 3 p. 1141-1156.
Datsenko and Wanner, (2000), Proc Natl Acad Sci USA., 97: 6640-6645.
Davis & Olsen., (2011), Mol. Biol. Evol., 28(1):211-221.
Dayhoff et al. (1978) “A model of evolutionary change in proteins,” in “Atlas of Protein Sequence and Structure,” Vol. 5, Suppl. 3 (ed. M. O. Dayhoff), pp. 345-352. Natl. Biomed. Res. Found., Washington, D.C.
Deml et al., (2011), J. Virol., 75(22): 10991-11001.
Durbin et al., (1998) Biological Sequence Analysis, Cambridge University Press.
Dykxhoorn et al., (1996), Gene, 177(1-2):133-6.
Ellman (1959), Archives of Biochemistry and Biophysics, 82 (1): 70-7.
Engvall and Perlman (1981), Immunochemistry, 8: 871-874.
Fallet et al., (2010), Biotechnology and Bioengineering. 107(1):124-33.
Goraj et al., (2019), Advancements of Microbiology, 58(3): 339-349.
Graf M, Bojak A, Deml L, Bieler K, Wolf H, Wagner R., (2000), J. Virol., 74(22): 10/22-10826.
He et al., (2015) Microb Cell Fact. 14(1), 55.
Henikoff and Henikoff (1992) Proc. Natl. Acad. Sci. USA, 89:10915-10919
Horton et al., (1990), Biotechniques., 8(5):528-35.
Kuhlmann and Bremer, (2002), Appl. Environ. Microbiol., 68:772-783
Miller, (1992) “A Short Course in Bacterial Genetics: A Laboratory Manual and Handbook for Escherichia coli and Related Bacteria”, Cold Spring Harbor Laboratory Press, Cold Spring Harbor, N.Y.
Needleman and Wunsch, (1970), J. Mol. Biol., 48(3), 443-453.
Ning et al., (2016), Metab Eng. 36:10-18.
Rui-Feng et al., (2017), World Journal of Microbiology and Biotechnology. 33(6):116.
Segel, Enzyme kinetics, (1993), John Wiley & Sons, pp. 44-54 and 100-112.
Schubert et al., (2007) Appl Environ Microbiol. 73(10): 3343-3347.
Schaefer et al., (1999), Anal. Biochem. 270: 88-96.
Strong et al., (2016), Bioresour Technol., 215:314-323

1. Microorganism genetically modified for the production of ectoine, wherein the microorganism comprises the following modifications:
expression of
heterologous gene ectA encoding a diaminobutyric acid acetyltransferase having at least 80% identity with SEQ ID NO: 1, SEQ ID NO: 2, SEQ ID NO: 3, SEQ ID NO: 4 or SEQ ID NO: 5, and
heterologous gene ectB encoding a diaminobutyric acid aminotransferase having at least 80% identity with SEQ ID NO: 6, SEQ ID NO: 7, SEQ ID NO: 8, SEQ ID NO: 9 or SEQ ID NO: 10, and
heterologous gene ectC encoding an ectoine synthase having at least 80% identity with SEQ ID NO: 11, SEQ ID NO: 12, SEQ ID NO: 13, SEQ ID NO: 14 or SEQ ID NO: 15,
deletion of pykA and pykF genes, and
at least a 50% reduction in citrate synthase enzyme activity as compared to an unmodified microorganism.

2. Microorganism of claim 1 further comprising at least a 75% reduction in citrate synthase enzyme activity.

3. Microorganism of claim 1, wherein the citrate synthase enzyme has at least 80% identity with the citrate synthase enzyme of SEQ ID NO: 18, SEQ ID NO: 19, SEQ ID NO: 20, or SEQ ID NO: 21, encoded by the gene gltA.

4. Microorganism of claim 1, wherein citrate synthase enzyme activity is reduced by placing the gltA gene encoding the citrate synthase under the control of promoter PgltA or a heterologous inducible promoter.

5. Microorganism of claim 3, wherein expression of the gltA gene, encoding the citrate synthase enzyme having at least 80% identity with the citrate synthase enzyme of SEQ ID NO: 18, SEQ ID NO: 19, SEQ ID NO: 20, or SEQ ID NO: 21, is under the control of promoter PgltA or under the control of a heterologous inducible promoter.

6. Microorganism of claim 1 further comprising a deletion of the gene ppc and an overexpression of the gene pck encoding a phosphoenolpyruvate carboxykinase having at least 80% identity with SEQ ID NO: 29, SEQ ID NO: 30, SEQ ID NO: 31, SEQ ID NO: 32 or SEQ ID NO: 33.

7. Microorganism of claim 1 further comprising an overexpression of an aspartate transaminase having at least 80% identity with SEQ ID NO: 35, SEQ ID NO: 36, SEQ ID NO: 37, SEQ ID NO: 38, and a glutamate dehydrogenase having at least 80% identity with SEQ ID NO: 39, SEQ ID NO: 40, SEQ ID NO: 41, SEQ ID NO: 42, SEQ ID NO: 43, SEQ ID NO: 44.

8. Microorganism of claim 1 further comprising a deletion of at least one gene selected from the group consisting of ackA-pta, adhE, frdABCD, ldhA, mgsA, pflAB, and mdh.

9. Microorganism of claim 1, wherein the microorganism has been genetically modified to be able to utilize sucrose as a carbon source, and wherein said microorganism further comprises the overexpression of:
the heterologous cscBKAR genes of E. coli EC3132, or
the heterologous scrKYABR genes of Salmonella sp.

10. Microorganism of claim 1, wherein the microorganism belongs to the family of the bacteria Enterobacteriaceae, Clostridiaceae, Bacillaceae, Streptomycetaceae, or Corynebacteriaceae, or to the family of yeasts Saccharomycetaceae.

11. Microorganism of claim 10, wherein the Enterobacteriaceae bacterium is Escherichia coli or Klebsiella pneumoniae, the Clostridiaceae bacterium is Clostridium acetobutylicum, the Corynebacteriaceae bacterium is Corynebacterium glutamicum, or the Saccharomycetaceae yeast is Saccharomyces cerevisiae.

12. Microorganism of claim 11, wherein the Enterobacteriaceae bacterium is Escherichia coli.

13. Method for the production of ectoine comprising the steps of:
a) culturing a microorganism genetically modified for the production of ectoine in an appropriate culture medium comprising a source of carbon and a source of nitrogen, and
b) recovering ectoine from the culture medium,
wherein the microorganism genetically modified for the production of ectoine comprises the following modifications:
expression of
heterologous gene ectA encoding a diaminobutyric acid acetyltransferase having at least 80% identity with SEQ ID NO: 1, SEQ ID NO: 2, SEQ ID NO: 3, SEQ ID NO: 4 or SEQ ID NO: 5, and
heterologous gene ectB encoding a diaminobutyric acid aminotransferase having at least 80% identity with SEQ ID NO: 6, SEQ ID NO: 7, SEQ ID NO: 8, SEQ ID NO: 9 or SEQ ID NO: 10, and
heterologous gene ectC encoding an ectoine synthase having at least 80% identity with SEQ ID NO: 11, SEQ ID NO: 12, SEQ ID NO: 13, SEQ ID NO: 14 or SEQ ID NO: 15,
deletion of pykA and pykF genes, and
at least a 50% reduction in citrate synthase enzyme activity as compared to an unmodified microorganism.

14. Method of claim 13, wherein the source of carbon is glycerol and/or glucose and/or sucrose.

15. Method of claim 13, wherein step b) comprises a step of filtration, desalination, cation exchange, liquid extraction, or distillation.

16. Microorganism of claim 5, wherein expression of the gltA gene is under the control of a promoter selected from the group consisting of a trc promoter, a tac promoter, a lac promoter, a tet promoter, a lambda P_{L} promoter, and a lambda P_{R} promoter.

17. Microorganism of claim 7 further comprising a deletion of at least one gene selected from the group consisting of ackA-pta, adhE, frdABCD, ldhA, mgsA, pflAB, and mdh.

18. Microorganism of claim 17, wherein the microorganism belongs to the family of the bacteria Enterobacteriaceae, Clostridiaceae, Bacillaceae, Streptomycetaceae, or Corynebacteriaceae, or to the family of yeasts Saccharomycetaceae.

19. Method for the production of ectoine of claim 13 wherein the microorganism genetically modified for the production of ectoine is a microorganism further comprising an overexpression of an aspartate transaminase having at least 80% identity with SEQ ID NO: 35, SEQ ID NO: 36, SEQ ID NO: 37, SEQ ID NO: 38, and a glutamate dehydrogenase having at least 80% identity with SEQ ID NO: 39, SEQ ID NO: 40, SEQ ID NO: 41, SEQ ID NO: 42, SEQ ID NO: 43, SEQ ID NO: 44, and a deletion of at least one gene selected from the group consisting of ackA-pta, adhE, frdABCD, ldhA, mgsA, pflAB, and mdh.

20. Method for the production of ectoine of claim 13 wherein the microorganism genetically modified for the production of ectoine is a microorganism further comprising an overexpression of an aspartate transaminase having at least 80% identity with SEQ ID NO: 35, SEQ ID NO: 36, SEQ ID NO: 37, SEQ ID NO: 38, and a glutamate dehydrogenase having at least 80% identity with SEQ ID NO: 39, SEQ ID NO: 40, SEQ ID NO: 41, SEQ ID NO: 42, SEQ ID NO: 43, SEQ ID NO: 44, and a deletion of at least one gene selected from the group consisting of ackA-pta, adhE, frdABCD, ldhA, mgsA, pflAB, and mdh, and wherein the microorganism belongs to the family of the bacteria Enterobacteriaceae, Clostridiaceae, Bacillaceae, Streptomycetaceae, or Corynebacteriaceae, or to the family of yeasts Saccharomycetaceae.


SEQUENCE LISTING (61 sequences)

SEQ ID NO: 1 - protein, 192 aa, Halomonas elongata
MNATTEPFTPSADLAKPSVADAVVGHEASPLFIRKPSPDDGWGIYELVKSCPPLDVNSAYAYLLLATQFRDSCAVATNEEGEIVGFVSGYVKSNAPDTYFLWQVAVGEKARGTGLARRLVEAVMTRPEMAEVHHLETTITPDNQASWGLFRRLADRWQAPLNSREYFSTDQLGGEHDPENLVRIGPFQTDQI

SEQ ID NO: 2 - protein, 193 aa, Pseudomonas stutzeri
MPTLKRNSINNPKGIVLSFPTVMLRRPTDGDGYNLHQLVARCQPLDTNSVYCNLLQCSDFADTAIAAENAQGELVGFISGYRPPSRPDTLFVWQVAVDSSMRGQGLALRMLLALTARVAREYGVRYMETTISPDNGASQALFKRAFDRLDANCTTRTLFARDTHFAGQHEDEVLYRAGPFTVSHLEEELKEHA

SEQ ID NO: 3 - protein, 192 aa, Chromohalobacter salexigens
MTPTTENFTPSADLARPSVADTVIGSAKKTLFIRKPTTDDGWGIYELVKACPPLDVNSGYAYLLLATQFRDTCAVATDEEGEIVGFVSGYVKRNAPDTYFLWQVAVGEKARGTGLARRLVEAVLMRPGMGDVRHLETTITPDNEASWGLFKRLADRWQAPLNSREYFSTGQLGGEHDPENLVRIGPFEPQQI

SEQ ID NO: 4 - protein, 189 aa, Bacillus halodurans
MQVQCNEKAFKGGFIINSQIATAPPKTLDTTITIGKPTVEDGAAMWELVNKSTLDTNSPYKYIMMCEYFAETCVVAKENERLVGFVTAFIPPEHQDVIFVWQIGVDSSQRGKGLASKLLQELISRDICSNVNYVEATVTPSNKASQALFQKLAREYNTQCEVSECFSEDLFPGDDHEAELTFRIGPLHP

SEQ ID NO: 5 - protein, 172 aa, Marinococcus halophilus
METKMTGTNGSVDSIVFDKPTVEDGADMWELVKNSTLDLNSSYKYIMMCEFFAETCVVAKENDELVGFVTAFIPPEKQDTVFVWQVGVDTSQRGKGLASRLLNALLERDVCENVLYLEATITPSNEASQALFKKLAQKRETEVTVSECFTEDLFPDDEHEEELTFRIGPFTK

SEQ ID NO: 6 - protein, 421 aa, Halomonas elongata
MQTQILERMESDVRTYSRSFPVVFTKARNARLTDEEGREYIDFLAGAGTLNYGHNNPHLKQALLDYIDSDGIVHGLDFWTAAKRDYLETLEEVILKPRGLDYKVHLPGPTGTNAVEAAIRLARVAKGRHNIVSFTNGFHGVTMGALATTGNRKFREATGGVPTQAASFMPFDGYLGSSTDTLDYFEKLLGDKSGGLDVPAAVIVETVQGEGGINVAGLEWLKRLESICRANDILLIIDDIQAGCGRTGKFFSFEHAGITPDIVTNSKSLSGYGLPFAHVLMRPELDKWKPGQYNGTFRGFNLAFATAAAAMRKYWSDDTFERDVQRKARIVEERFGKIAAWLSENGIEASERGRGLMRGIDVGSGDIADKITHQAFENGLIIETSGQDGEVVKCLCPLTIPDEDLVEGLDILETSTKQAFS

SEQ ID NO: 7 - protein, 425 aa, Pseudomonas stutzeri
MKTFELNESRVRSYCRSFPVVFKQAQGAELVTQDGKRYIDFLAGAGTLNYGHNHPVLKQALLEYIESDGITHGLDMYTEAKERFLETFNRLILEPRGMGDYRMQFTGPTGTNAVEAAMKLARKVTGRNNIISFTNGFHGCSIGALAATGNQHHRGGSGISLTDVSRMPYANYFGDKTNTIGMMDKLLSDPSSGIDKPAAVIVEVVQGEGGLNTASAEWMRKLEKLCRKHEMLLIVDDIQAGCGRTGTFFSFEEMGIQPDIVTLSKSLSGYGLPFAMVLLRQELDQWKPGEHNGTFRGNNHAFVTAAAAVEHFWQNDAFANSVKAKGKRIADGMQRIIRRHGPDSLFLKGRGMMIGISCPDGEIAAAVCRHAFENGLVIETSGAHSEVVKCLCPLIISDEQIDKALSILDKAFAAVMSEQTENQAS

SEQ ID NO: 8 - protein, 423 aa, Chromohalobacter salexigens
MQTQILERMESEVRTYSRSFPTVFTEAKGARLHAEDGNQYIDFLAGAGTLNYGHNHPKLKQALADYIASDGIVHGLDMWSAAKRDYLETLEEVILKPRGLDYKVHLPGPTGTNAVEAAIRLARNAKGRHNIVTFTNGFHGVTMGALATTGNRKFREATGGIPTQGASFMPFDGYMGEGVDTLSYFEKLLGDNSGGLDVPAAVIIETVQGEGGINPAGIPWLQRLEKICRDHDMLLIVDDIQAGCGRTGKFFSFEHAGITPDIVTNSKSLSGFGLPFAHVLMRPELDIWKPGQYNGTFRGFNLAFVTAAAAMRHFWSDDTFERDVQRKGRVVEDRFQKLASFMTEKGHPASERGRGLMRGLDVGDGDMADKITAQAFKNGLIIETSGHSGQVIKCLCPLTITDEDLVGGLDILEQSVKEVFGQA

SEQ ID NO: 9 - protein, 427 aa, Bacillus halodurans
MSQTDMNVFEQLESEVRSYCRSFPTVFTKAKGYKMWDEAGKEYIDFFSGAGALNYGHNDEKMKKALVDYIMDDGITHSLDMATTPKGKFLQKFHDVILKPRNLDYKVMFPGPTGTNTVESALKLARKVTGRTDIISFTNGFHGMTIGSLSVTGNSFKRKGAGIPLTNVVTMPYDNFVSESLDTLDYLERFLEDGGSGVEIPAAMILETVQGEGGINAARTEWLQRVEKICKRWGILLIIDDVQAGVGRTGTFFSFEDAGITPDIVCLSKSIGGFGLPLAITLFRPELDIWAPGEHNGTFRGNNHAFVTATEALSYWEDDSFEKDIQEKSATISDFLVKLVTEYPEIKGEVKGKGFMVGIASDVEGFASKVTEEAFSRGLIMETSGPNDEVFKLFPPLTIDDEGLEKGLAIIEESIKALVETKELVMQ

SEQ ID NO: 10 - protein, 427 aa, Marinococcus halophilus
MMQNDLSVFNEYESEVRSYVRGFPTVFHQAKGYKLWDLDGKEYVDFFSGAGALNYGHNDENMKQKLLTYIQEDGVTHSLDMATKAKGEFIDAFQNIILKPRNMDYKIMFPGPTGANSVESALKLARKVTGRTNVVSFTNGFHGMTIGALSVTGNKFKRNGAGMPLSNTSTLPYDQFLKESNNSIEYIENFLDNGGSGLDKPAAFIVETVQGEGGLNAASSEWLRSIEKICRERDIKLILDDVQAGVGRTGTFFSFEPAGIKPDFVCLSKSIGGNGSPLAITLVAPEYDKFAPGEHNGTFRGNNFAFVTGTEALNYWKDDRLEKNVQEKSERITSFLDDMIKKHPEMKGVRKGRGFMQGIMSPIEDLADNIAGRCFEHGLIMETAGAEDEVFKLFPPITIDDEGLERGLSILQQAIEEVTAESNLVAK

SEQ ID NO: 11 - protein, 137 aa, Halomonas elongata
MIVRNLEEARQTDRLVTAENGNWDSTRLSLAEDGGNCSFHITRIFEGTETHIHYKHHFEAVYCIEGEGEVETLADGKIWPIKPGDIYILDQHDEHLLRASKTMHLACVFTPGLTGNEVHREDGSYAPADEADDQKPL

SEQ ID NO: 12 - protein, 133 aa, Pseudomonas stutzeri
MIVRTLAECEKTDRKVHSQTGTWDSTRMLLKDDKVGFSFHITTIYAGSETHIHYQNHFESVYCISGNGEIETIADGKIYKIEPGTLYVLDKHDEHLLRGGSEDMKLACVFNPPLNGREVHDESGVYPLEAETV

SEQ ID NO: 13 - protein, 130 aa, Chromohalobacter salexigens
MIVRNLEECRKTERFVEAENGNWDSTRLVLADDNVGFSFNITRIHPGTETHIHYKHHFEAVFCYEGEGEVETLADGKIHPIKAGDMYLLDQHDEHLLRGKEKGMTVACVFNPALTGREVHREDGSYAPVD

SEQ ID NO: 14 - protein, 129 aa, Bacillus halodurans
MKVVKLEDVIGTEQEVKGENWTSRRLLLKKDGMGYSVHDTIIKAGTETHIWYQNHLEAVYCIEGEGEVETVKDGKVWPIKANEIYALDEHDEHLLRAKTDMRMVCVFNPPITGKETHDENGVYPLVDDE

SEQ ID NO: 15 - protein, 129 aa, Marinococcus halophilus
MKVIKLEDLLGTEREVDDGNWVSRRFIMKDDNMGYSVNDTIIRAGTETHIWYQNHLETVYCIEGDGEIETLSDNKVYQLEPGVLYALDKNDEHMLRGGSKDMRMVCVFNPPLSGREVHDENGVYPADLD

SEQ ID NO: 16 - DNA, 1443 bp, Escherichia coli
atgtccagaaggcttcgcagaacaaaaatcgttaccacgttaggcccagcaacagatcgcgataataatcttgaaaaagttatcgcggcgggtgccaacgttgtacgtatgaacttttctcacggctcgcctgaagatcacaaaatgcgcgcggataaagttcgtgagattgccgcaaaactggggcgtcatgtggctattctgggtgacctccaggggcccaaaatccgtgtatccacctttaaagaaggcaaagttttcctcaatattggggataaattcctgctcgacgccaacctgggtaaaggtgaaggcgacaaagaaaaagtcggtatcgactacaaaggcctgcctgctgacgtcgtgcctggtgacatcctgctgctggacgatggtcgcgtccagttaaaagtactggaagttcagggcatgaaagtgttcaccgaagtcaccgtcggtggtcccctctccaacaataaaggtatcaacaaacttggcggcggtttgtcggctgaagcgctgaccgaaaaagacaaagcagacattaagactgcggcgttgattggcgtagattacctggctgtctccttcccacgctgtggcgaagatctgaactatgcccgtcgcctggcacgcgatgcaggatgtgatgcgaaaattgttgccaaggttgaacgtgcggaagccgtttgcagccaggatgcaatggatgacatcatcctcgcctctgacgtggtaatggttgcacgtggcgacctcggtgtggaaattggcgacccggaactggtcggcattcagaaagcgttgatccgtcgtgcgcgtcagctaaaccgagcggtaatcacggcgacccagatgatggagtcaatgattactaacccgatgccgacgcgtgcagaagtcatggacgtagcaaacgccgttctggatggtactgacgctgtgatgctgtctgcagaaactgccgctgggcagtatccgtcagaaaccgttgcagccatggcgcgcgtttgcctgggtgcggaaaaaatcccgagcatcaacgtttctaaacaccgtctggacgttcagttcgacaatgtggaagaagctattgccatgtcagcaatgtacgcagctaaccacctgaaaggcgttacggcgatcatcaccatgaccgaatcgggtcgtaccgcgctgatgacctcccgtatcagctctggtctgccaattttcgccatgtcgcgccatgaacgtacgctgaacctgactgctctctatcgtggcgttacgccggtgcactttgatagcgctaatgacggcgtagcagctgccagcgaagcggttaatctgctgcgcgataaaggttacttgatgtctggtgacctggtgattgtcacccagggcgacgtgatgagtaccgtgggttctactaataccacgcgtattttaacggtagagtaa

SEQ ID NO: 17 - DNA, 1413 bp, Escherichia coli
atgaaaaagaccaaaattgtttgcaccatcggaccgaaaaccgaatctgaagagatgttagctaaaatgctggacgctggcatgaacgttatgcgtctgaacttctctcatggtgactatgcagaacacggtcagcgcattcagaatctgcgcaacgtgatgagcaaaactggtaaaaccgccgctatcctgcttgataccaaaggtccggaaatccgcaccatgaaactggaaggcggtaacgacgtttctctgaaagctggtcagacctttactttcaccactgataaatctgttatcggcaacagcgaaatggttgcggtaacgtatgaaggtttcactactgacctgtctgttggcaacaccgtactggttgacgatggtctgatcggtatggaagttaccgccattgaaggtaacaaagttatctgtaaagtgctgaacaacggtgacctgggcgaaaacaaaggtgtgaacctgcctggcgtttccattgctctgccagcactggctgaaaaagacaaacaggacctgatctttggttgcgaacaaggcgtagactttgttgctgcttcctttattcgtaagcgttctgacgttatcgaaatccgtgagcacctgaaagcgcacggcggcgaaaacatccacatcatctccaaaatcgaaaaccaggaaggcctcaacaacttcgacgaaatcctcgaagcctctgacggcatcatggttgcgcgtggcgacctgggtgtagaaatcccggtagaagaagttatcttcgcccagaagatgatgatcgaaaaatgtatccgtgcacgtaaagtcgttatcactgcgacccagatgctggattccatgatcaaaaacccacgcccgactcgcgcagaagccggtgacgttgcaaacgccatcctcgacggtactgacgcagtgatgctgtctggtgaatccgcaaaaggtaaatacccgctggaagcggtttctatcatggcgaccatctgcgaacgtaccgaccgcgtgatgaacagccgtctcgagttcaacaatgacaaccgtaaactgcgcattaccgaagcggtatgccgtggtgccgttgaaactgctgaaaaactggatgctccgctgatcgtggttgctactcagggcggtaaatctgctcgcgcagtacgtaaatacttcccggatgccaccatcctggcactgaccaccaacgaaaaaacggctcatcagttggtactgagcaaaggcgttgtgccgcagcttgttaaagagatcacttctactgatgatttctaccgtctgggtaaagaactggctctgcagagcggtctggcacacaaaggtgacgttgtagttatggtttctggtgcactggtaccgagcggcactactaacaccgcatctgttcacgtcctgtaa

SEQ ID NO: 18 - protein, 427 aa, Escherichia coli
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFRLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 19 - protein, 437 aa, Corynebacterium glutamicum [sequence omitted]

SEQ ID NO: 20 - protein, 377 aa, Thermus thermophilus
MEVARGLEGVLFTESRMCYIDGQQGKLYYYGIPIQELAEKSSFEETTFLLLHGRLPRRQELEEFSAALARRRALPAHLLESFKRYPVSAHPMSFLRTAVSEFGMLDPTEGDISREALYEKGLDLIAKFATIVAANKRLKEGKEPIPPREDLSHAANFLYMANGVEPSPEQARLMDAALILHAEHGFNASTFTAIAAFSTETDLYSAITAAVASLKGPRHGGANEAVMRMIQEIGTPERAREWVREKLAKKERIMGMGHRVYKAFDPRAGVLEKLARLVAEKHGHSKEYQILKIVEEEAGKVLNPRGIYPNVDFYSGVVYSDLGFSLEFFTPIFAVARISGWVGHILEYQELDNRLLRPGAKYVGELDVPYVPLEARE

SEQ ID NO: 21 - protein, 366 aa, Bacillus subtilis
MVHYGLKGITCVETSISHIDGEKGRLIYRGHHAKDIALNHSFEEAAYLILFGKLPSTEELQVFKDKLAAERNLPEHIERLIQSLPNNMDDMSVLRTVVSALGENTYTFHPKTEEAIRLIAITPSIIAYRKRWTRGEQAIAPSSQYGHVENYYYMLTGEQPSEAKKKALETYMILATEHGMNASTFSARVTLSTESDLVSAVTAALGTMKGPLHGGAPSAVTKMLEDIGEKEHAEAYLKEKLEKGERLMGFGHRVYKTKDPRAEALRQKAEEVAGNDRDLDLALHVEAEAIRLLEIYKPGRKLYTNVEFYAAAVMRAIDFDDELFTPTFSASRMVGWCAHVLEQAENNMIFRPSAQYTGAIPEEVLS

SEQ ID NO: 22 - DNA, 708 bp, Escherichia coli
gaataacgcccacatgctgttcttattattccctggggactacgggcacagaggttaactttctgttacctggagacgtcgggatttccttcctccggtctgcttgcgggtcagacagcgtcctttctataactgcgcgtcatgcaaaacactgcttccagatgcgaaaacgacacgttacaacgctgggtggctcgggattgcagggtgttccggagacctggcggcagtataggctgttcacaaaatcattacaattaacctacatatagtttgtcgggttttatcctgaacagtgatccaggtcacgataacaacatttatttaatttttaatcatctaatttgacaatcattcaacaaagttgttacaaacattaccaggaaaagcatataatgcgtaaaagttatgaagtcggtatttcacctaagattaacttatgtaacagtgtggaagtattgaccaattcattcgggacagttattagtggtagacaagtttaataattcggattgctaagtacttgattcgccatttattcgtcatcaatggatcctttacctgcaagcgcccagagctctgtacccaggttttcccctctttcacagagcggcgagccaaataaaaaacgggtaaagccaggttgatgtgcgaaggcaaatttaagttccggcagtcttacgcaataaggcgctaaggagaccttaa

SEQ ID NO: 23 - DNA, 1445 bp, Artificial
  Ptrc regulation system
acaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaa

SEQ ID NO: 24 - DNA, 1330 bp, Artificial
  Ptac regulation system
gacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgactgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaa

SEQ ID NO: 25 - DNA, 1445 bp, Artificial
  Plac regulation system
ctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtaggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgtttacactttatgcttccggctcgtatgttgtgtggaattgtgagcggataaca

SEQ ID NO: 26 - DNA, 1629 bp, Artificial
  Ptet regulation system
tgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagaccatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagggggcgcgagatcccccgggctgcaggaattcctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagct

SEQ ID NO: 27 - DNA, 900 bp, Artificial
  PL regulation system
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgcagctatgaccatgattacgccaagcttgcatgcctgcaggtcgactataaaaaacatacagataaccatctgcggtgataaattatctctggcggtgttgacataaataccactggcggtgatactgagcacatcaac

SEQ ID NO: 28 - DNA, 796 bp, Artificial
  PR regulation system
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgactattttacctctggcggtgataatggttgc

SEQ ID NO: 29 - protein, 540 aa, Escherichia coli
MRVNNGLTPQELEAYGISDVHDIVYNPSYDLLYQEELDPSLTGYERGVLTNLGAVAVDTGIFTGRSPKDKYIVRDDTTRDTFWWADKGKGKNDNKPLSPETWQHLKGLVTRQLSGKRLFVVDAFCGANPDTRLSVRFITEVAWQAHFVKNMFIRPSDEELAGFKPDFIVMNGAKCTNPQWKEQGLNSENFVAFNLTERMQLIGGTWYGGEMKKGMFSMMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGTIDFDDGSKTENTRVSYPIYHIDNIVKPVSKAGHATKVIFLTADAFGVLPPVSRLTADQTQYHFLSGFTAKLAGTERGITEPTPTFSACFGAAFLSLHPTQYAEVLVKRMQAAGAQAYLVNTGWNGTGKRISIKDTRAIIDAILNGSLDNAETFTLPMFNLAIPTELPGVDTKILDPRNTYASPEQWQEKAETLAKLFIDNFDKYTDTPAGAALVAAGPKL

SEQ ID NO: 30 - protein, 532 aa, Anaerobiospirillum succiniciproducens
MSLSESLAKYGITGATNIVHNPSHEELFAAETQASLEGFEKGTVTEMGAVNVMTGVYTGRSPKDKFIVKNEASKEIWWTSDEFKNDNKPVTEEAWAQLKALAGKELSNKPLYVVDLFCGANENTRLKIRFVMEVAWQAHFVTNMFIRPTEEELKGFEPDFVVLNASKAKVENFKELGLNSETAVVFNLAEKMQIILNTWYGGEMKKGMFSMMNFYLPLQGIAAMHCSANTDLEGKNTAIFFGLSGTGKTTLSTDPKRLLIGDDEHGWDDDGVFNFEGGCYAKVINLSKENEPDIWGAIKRNALLENVTVDANGKVDFADKSVTENTRVSYPIFHIKNIVKPVSKAPAAKRVIFLSADAFGVLPPVSILSKEQTKYYFLSGFTAKLAGTERGITEPTPTFSSCFGAAFLTLPPTKYAEVLVKRMEASGAKAYLVNTGWNGTGKRISIKDTRGIIDAILDGSIDTANTATIPYFNFTVPTELKGVDTKILDPRNTYADASEWEVKAKDLAERFQKNFKKFESLGGDLVKAGPQL

SEQ ID NO: 31 - protein, 472 aa, Trypanosoma cruzi
MPPTIHRNLLSPELVQWALKIEKDSRLTARGALAVMSYAKTGRSPLDKRIVDTDDVRENVDWGKVNMKLSEESFARVRKIAKEFLDTREHLFVVDCFAGHDERYRLKVRVFTTRPYHALFMRDMLIVPTPEELATFGEPDYVIYNAGECKADPSIPGLTSTTCVALNFKTREQVILGTEYAGEMKKGILTVMFELMPQMNHLCMHASANVGKQGDVTVFFGLSGTGKTTLSADPHRNLIGDDEHVWTDRGVFNIEGGCYAKAIGLNPKTEKDIYDAVRFGAVAENCVLDKRTGEIDFYDESICKNTRVAYPLSHIEGALSKAIAGHPKNVIFLTNDAFGVMPPVARLTSAQAMFWFVMGYTANVPGVEAGGTRTARPIFSSCFGGPFLVRHATFYGEQLAEKMQKHNSRVWLLNTGYAGGRADRGAKRMPLRVTRAIIDAIHDGTLDRTEYEEYPGWACTSRSTSPKCRSIC

SEQ ID NO: 32 - protein, 538 aa, Actinobacillus succinogenes
MTDLNKLVKELNDLGLTDVKEIVYNPSYEQLFEEETKPGLEGFDKGTLTTLGAVAVDTGIFTGRSPKDKYIVCDETTKDTVWWNSEAAKNDNKPMTQETWKSLRELVAKQLSGKRLFVVEGYCGASEKHRIGVRMVTEVAWQAHFVKNMFIRPTDEELKNFKADFTVLNGAKCTNPNWKEQGLNSENFVAFNITEGIQLIGGTWYGGEMKKGMFSMMNYFLPLKGVASMHCSANVGKDGDVAIFFGLSGTGKTTLSTDPKRQLIGDDEHGWDESGVFNFEGGCYAKTINLSQENEPDIYGAIRRDALLENVVVRADGSVDFDDGSKTENTRVSYPIYHIDNIVRPVSKAGHATKVIFLTADAFGVLPPVSKLTPEQTEYYFLSGFTAKLAGTERGVTEPTPTFSACFGAAFLSLHPIQYADVLVERMKASGAEAYLVNTGWNGTGKRISIKDTRGIIDAILDGSIEKAEMGELPIFNLAIPKALPGVDPAILDPRDTYADKAQWQVKAEDLANRFVKNFVKYTANPEAAKLVGAGPKA

SEQ ID NO: 33 - protein, 549 aa, Saccharomyces cerevisiae
MSPSKMNATVGSTSEVEQKIRQELALSDEVTTIRRNAPAAVLYEDGLKENKTVISSSGALIAYSGVKTGRSPKDKRIVEEPTSKDEIWWGPVNKPCSERTWSINRERAADYLRTRDHIYIVDAFAGWDPKYRIKVRVVCARAYHALFMTNMLIRPTEEELAHFGEPDFTVWNAGQFPANLHTQDMSSKSTIEINFKAMEMIILGTEYAGEMKKGIFTVMFYLMPVHHNVLTLHSSANQGIQNGDVTLFFGLSGTGKTTLSADPHRLLIGDDEHCWSDHGVFNIEGGCYAKCINLSAEKEPEIFDAIKFGSVLENVIYDEKSHVVDYDDSSITENTRCAYPIDYIPSAKIPCLADSHPKNIILLTCDASGVLPPVSKLTPEQVMYHFISGYTSKMAGTEQGVTEPEPTFSSCFGQPFLALHPIRYATMLATKMSQHKANAYLINTGWTGSSYVSGGKRCPLKYTRAILDSIHDGSLANETYETLPIFNLQVPTKVNGVPAELLNPAKNWSQGESKYRGAVTNLANLFVQNFKIYQDRATPDVLAAGPQFE

SEQ ID NO: 34 - DNA, 2652 bp, Escherichia coli
atgaacgaacaatattccgcattgcgtagtaatgtcagtatgctcggcaaagtgctgggagaaaccatcaaggatgcgttgggagaacacattcttgaacgcgtagaaactatccgtaagttgtcgaaatcttcacgcgctggcaatgatgctaaccgccaggagttgctcaccaccttacaaaatttgtcgaacgacgagctgctgcccgttgcgcgtgcgtttagtcagttcctgaacctggccaacaccgccgagcaataccacagcatttcgccgaaaggcgaagctgccagcaacccggaagtgatcgcccgcaccctgcgtaaactgaaaaaccagccggaactgagcgaagacaccatcaaaaaagcagtggaatcgctgtcgctggaactggtcctcacggctcacccaaccgaaattacccgtcgtacactgatccacaaaatggtggaagtgaacgcctgtttaaaacagctcgataacaaagatatcgctgactacgaacacaaccagctgatgcgtcgcctgcgccagttgatcgcccagtcatggcataccgatgaaatccgtaagctgcgtccaagcccggtagatgaagccaaatggggctttgccgtagtggaaaacagcctgtggcaaggcgtaccaaattacctgcgcgaactgaacgaacaactggaagagaacctcggctacaaactgcccgtcgaatttgttccggtccgttttacttcgtggatgggcggcgaccgcgacggcaacccgaacgtcactgccgatatcacccgccacgtcctgctactcagccgctggaaagccaccgatttgttcctgaaagatattcaggtgctggtttctgaactgtcgatggttgaagcgacccctgaactgctggcgctggttggcgaagaaggtgccgcagaaccgtatcgctatctgatgaaaaacctgcgttctcgcctgatggcgacacaggcatggctggaagcgcgcctgaaaggcgaagaactgccaaaaccagaaggcctgctgacacaaaacgaagaactgtgggaaccgctctacgcttgctaccagtcacttcaggcgtgtggcatgggtattatcgccaacggcgatctgctcgacaccctgcgccgcgtgaaatgtttcggcgtaccgctggtccgtattgatatccgtcaggagagcacgcgtcataccgaagcgctgggcgagctgacccgctacctcggtatcggcgactacgaaagctggtcagaggccgacaaacaggcgttcctgatccgcgaactgaactccaaacgtccgcttctgccgcgcaactggcaaccaagcgccgaaacgcgcgaagtgctcgatacctgccaggtgattgccgaagcaccgcaaggctccattgccgcctacgtgatctcgatggcgaaaacgccgtccgacgtactggctgtccacctgctgctgaaagaagcgggtatcgggtttgcgatgccggttgctccgctgtttgaaaccctcgatgatctgaacaacgccaacgatgtcatgacccagctgctcaatattgactggtatcgtggcctgattcagggcaaacagatggtgatgattggctattccgactcagcaaaagatgcgggagtgatggcagcttcctgggcgcaatatcaggcacaggatgcattaatcaaaacctgcgaaaaagcgggtattgagctgacgttgttccacggtcgcggcggttccattggtcgcggcggcgcacctgctcatgcggcgctgctgtcacaaccgccaggaagcctgaaaggcggcctgcgcgtaaccgaacagggcgagatgatccgctttaaatatggtctgccagaaatcaccgtcagcagcctgtcgctttataccggggcgattctggaagccaacctgctgccaccgccggagccgaaagagagctggcgtcgcattatggatgaactgtcagtcatctcctgcgatgtctaccgcggctacgtacgtgaaaacaaagattttgtgccttacttccgctccgctacgccggaacaagaactgggcaaactgccgttgggttcacgtccggcgaaacgtcgcccaaccggcggcgtcgagtcactacgcgccattccgtggatcttcgcctggacgcaaaaccgtctgatgctccccgcctggctgggtgcaggtacggcgctgcaaaaagtggtcgaagacggcaaacagagcgagctggaggctatgtgccgcgattggccattcttctcgacgcgtctcggcatgctggagatggtcttcgccaaagcagacctgtggctggcggaatactatgaccaacgcctggtagacaaagcactgtggccgttaggtaaagagttacgcaacctgcaagaagaagacatcaaagtggtgctggcgattgccaacgattcccatctgatggccgatctgccgtggattgcagagtctattcagctacggaatatttacaccgacccgctgaacgtattgcaggccgagttgctgcaccgctcccgccaggcagaaaaagaaggccaggaaccggatcctcgcgtcgaacaagcgttaatggtcactattgccgggattgcggcaggtatgcgtaataccggctaa

SEQ ID NO: 35 - protein, 396 aa, Escherichia coli
MFENITAAPADPILGLADLFRADERPGKINLGIGVYKDETGKTPVLTSVKKAEQYLLENETTKNYLGIDGIPEFGRCTQELLFGKGSALINDKRARTAQTPGGTGALRVAADFLAKNTSVKRVWVSNPSWPNHKSVFNSAGLEVREYAYYDAENHTLDFDALINSLNEAQAGDVVLFHGCCHNPTGIDPTLEQWQTLAQLSVEKGWLPLFDFAYQGFARGLEEDAEGLRAFAAMHKELIVASSYSKNFGLYNERVGACTLVAADSETVDRAFSQMKAAIRANYSNPPAHGASVVATILSNDALRAIWEQELTDMRQRIQRMRQLFVNTLQEKGANRDFSFIIKQNGMFSFSGLTKEQVLRLREEFGVYAVASGRVNVAGMTPDNMAPLCEAIVAVL

SEQ ID NO: 36 - protein, 432 aa, Corynebacterium glutamicum
MRRYAVMSSVSLQDFDAERIGLFHEDIKRKFDELKSKNLKLDLTRGKPSSEQLDFADELLALPGKGDFKAADGTDVRNYGGLDGIVDIRQIWADLLGVPVEQVLAGDASSLNIMFDVISWSYIFGNNDSVQPWSKEETVKWICPVPGYDRHFSITERFGFEMISVPMNEDGPDMDAVEELVKNPQVKGMWVVPVFSNPTGFTVTEDVAKRLSAMETAAPDFRVVWDNAYAVHTLTDEFPEVIDIVGLGEAAGNPNRFWAFTSTSKITLAGAGVSFFLTSAENRKWYTGHAGIRGIGPNKVNQLAHARYFGDAEGVRAVMRKHAASLAPKFNKVLEILDSRLAEYGVAQWTVPAGGYFISLDVVPGTASRVAELAKEAGIALTGAGSSYPLRQDPENKNLRLAPSLPPVEELEVAMDGVATCVLLAAAEHYAN

SEQ ID NO: 37 - protein, 385 aa, Thermus thermophilus [sequence omitted]

SEQ ID NO: 38 - protein, 436 aa, Bacillus subtilis
MNDAAKELNRTLSEENPHVLHMLSDLGRELFYPKGVLTQSAEAKAKAGKYNATIGIATSQGESMHFSHIQETLSAYNPDDIYDYAPPQGKEPLRQEWLKKMRLENPSLAGKDISTPIVTNALTHGLSIAADLFVNEGDTLLLPDKYWGNYNFIFGVRRKASIETYPLFQQDGRFNAAGLSELLKKQEEKAIVVLNFPNNPTGYTPGEEEASEIVSVILEAAEAGKEIVVLVDDAYYNLFYDETAIQESIFSKLAQVHDRVLCVKIDGATKENYAWGFRVGFITYSTKSEKALRVLEEKTKGIIRGTISSAPHPSQTFMLRAMQSPEYEKEKSLKYNIMKKRADKVKAVLAENKHYEDVWTPYPFNSGYFMCVRLKDINAGELRVSLLEKRGIGTISINETDLRIAFSCVEEEHIADLFEEIYQEAKQLQKQAEISG

SEQ ID NO: 39 - protein, 424 aa, Bacillus subtilis
MSAKQVSKDEEKEALNLFLSTQTIIKEALRKLGYPGDMYELMKEPQRMLTVRIPVKMDNGSVKVFTGYRSQHNDAVGPTKGGVRFHPEVNEEEVKALSIWMTLKCGIANLPYGGGKGGIICDPRTMSFGELERLSRGYVRAISQIVGPTKDIPAPDVYTNSQIMAWMMDEYSRLREFDSPGFITGKPLVLGGSQGRETATAQGVTICIEEAVKKKGIKLQNARIIIQGFGNAGSFLAKFMHDAGAKVIGISDANGGLYNPDGLDIPYLLDKRDSFGMVTNLFTDVITNEELLEKDCDILVPAAISNQITAKNAHNIQASIVVEAANGPTTIDATKILNERGVLLVPDILASAGGVTVSYFEWVQNNQGYYWSEEEVAEKLRSVMVSSFETIYQTAATHKVDMRLAAYMTGIRKSAEASRFRGWV

SEQ ID NO: 40 - protein, 450 aa, Clostridium symbiosum
MSKYVDRVIAEVEKKYADEPEFVQTVEEVLSSLGPVVDAHPEYEEVALLERMVIPERVIEFRVPWEDDNGKVHVNTGYRVQFNGAIGPYKGGLRFAPSVNLSIMKFLGFEQAFKDSLTTLPMGGAKGGSDFDPNGKSDREVMRFCQAFMTELYRHIGPDIDVPAGDLGVGAREIGYMYGQYRKIVGGFYNGVLTGKARSFGGSLVRPEATGYGSVYYVEAVMKHENDTLVGKTVALAGFGNVAWGAAKKLAELGAKAVTLSGPDGYIYDPEGITTEEKINYMLEMRASGRNKVQDYADKFGVQFFPGEKPWGQKVDIIMPCATQNDVDLEQAKKIVANNVKYYIEVANMPTTNEALRFLMQQPNMVVAPSKAVNAGGVLVSGFEMSQNSERLSWTAEEVDSKLHQVMTDIHDGSAAAAERYGLGYNLVAGANIVGFQKIADAMMAQGIAW

SEQ ID NO: 41 - protein, 421 aa, Peptoniphilus asaccharolyticus
MTDTLNPLVAAQEKVRIACEKLGCDPAVYELLKEPQRVIEISIPVKMDDGTVKVFKGWRSAHSSAVGPSKGGVRFHPNVNMDEVKALSLWMTFKGGALGLPYGGGKGGICVDPAELSERELEQLSRGWVRGLYKYLGDRIDIPAPDVNTNGQIMSWFVDEYVKLNGERMDIGTFTGKPVAFGGSEGRNEATGFGVAVVVRESAKRFGIKMEDAKIAVQGFGNVGTFTVKNIERQGGKVCAIAEWDRNEGNYALYNENGIDFKELLAYKEANKTLIGFPGAERITDEEFWTKEYDIIVPAALENVITGERAKTINAKLVCEAANGPTTPEGDKVLTERGINLTPDILTNSGGVLVSYYEWVQNQYGYYWTEAEVEEKQEADMMKAIKGVFAVADEYNVTLREAVYMYAIKSIDVAMKLRGWY

SEQ ID NO: 42 - protein, 424 aa, Pyrobaculum calidifontis
MSTTYIVSDFLINTLLTIKRGVELAGLPPEFYEALEKPKRILVVNIPVKMDDGKIKYFEGYRVQHNDALGPFKGGIRFHPEVTLADDIALAMLMTLKNSLAGLPYGGAKGAVRVDPRRLSRRELEELARGYARAVAPLIGEQLDIPAPDVGTDSQVMAWMVDEYSRLVGRNAPAVFTSKPPELWGNPVREYSTGFGVAVAAREVAKRLWGGIVGKTAAVQGLGNVGRWAAYWLEKMGAKVVAVSDVNGVVYRERGLDVDLIRETKAKGPQLLEMISQKNGVEIVKNPDQIFSLDVDILVPAAIENVVREDNVDGVRARLVVEGANGPTTPGAERRLYERGVVVVPDILANAGGVIMSYLEWVENLQWLFWDEEETRRRLEAIMSNNVARVYARWEKEKSWTMRDAAVVTALERIYNAMKTRGWI

SEQ ID NO: 43 - protein, 434 aa, Burkholderia thailandensis
MSSQSQSPSVAQSIPSYLHADDLGPWGNYLQQVDRVAPYLGSLSRWIETLKRPKRILIVDVPIELDNGTVAHFEGYRVQHNVSRGPGKGGVRYHQDVTLSEVMALSAWMSVKNAAVNVPYGGAKGGIRVDPRKLSRGELERVTRRYTSEIGIIIGPNTDIPAPDVNTNEQIMAWMMDTYSMNQGQTATGVVTGKPISLGGSLGRKEATGRGVFVVGCEAAKKKGVEIEGARIAVQGFGNVGGIAAKLFQEAGAKVIAVQDHTGTIHQPAGVDTAKLLDHVGRTGGVAGFEGAEPMPNDEFWTVETEILIPAALENQITEKNASKIRTKIIVEGANGPTTTAADDILSANGVLVIPDVIANAGGVTVSYFEWVQDFSSFFWTEDEINHRLERVMREAFAGVWAVAEEHKVSVRTAAFIVACKRILMAREMRGLYP

SEQ ID NO: 44 - protein, 1042 aa, Dictyostelium discoideum
MLNQNLTISQEIAAQPKTQYFTKEDGDALANLLESNPYKEQAVEVKSLLKSERLIESSRVEPEVDWFYCKLGLDSNYFDSTPSIVIARHILSLYAAKMVSHATGAKLEVHLHSKNEGSATFITPSNPGKRDSPAMMIEHAIESHYFGEGYHQDQQLLSPQQVAVAPFPVSPKPPTGTNLPPHGFRLACYRTTGTVSNSSPVHLRLYYLTKPVFPQATNDLSASKNDEILATETDLFKIGDISFIEKSSELTKKIYQEVMNEVVGKQGPVIKHYPYQTNGARLVIAYRRGSTHSYWSAIGELYHFHQMYATHKYVEQFSNGITIYSIYLRPLHPDVDINTKISKIAEQASLVYVLPRTSLTPLFLSHQLSFPEVTYAYVCWKFAYQFLNRYATEYSALAAAIGDDSTKQSMLAQLKTRLSKDTFTEGRVRDAVLQYPELIKILYQDFEKFHFSGSNSNNTQKYDVQHGSEILASIKKTVNNELDSQIFSAILSFNRHLLKTNFYKQTKTALSFRLDPGFLSTKEYVSTPYAVFFVVGSEFRGFHIRFRDISRGGIRIIRSGNSTQYDHNSSSLFDENYNLANTQQSKNKDIAEGGSKGTILLSADHQSKAEVAFHKYIDGLLDLLLPNHEIVDHFAKPEILFLGPDEGTADFMNWASSHAKDRGAHFWKAFTTGKSLSRGGIPHDLYGMTTRSIHQYVLGTLAKLGRNEADCTKFQTGGPDGDLGSNEIKISKDKTIGIVDGSGVLLDPQGLNRDEIGRLASKRQMARYFDKSKLSPQGFFVDVAENDVKLPNGDIVESGLIFRNNFHLNPLCNADIFVPCGGRPESVQLTNVDKMFTATGESRFPIIVEGANLFFTQKARLMIEEKGAIIFKDASANKGGVTSSSLEVLAALALNDEEFDRHMCVKDNVVPEFYENYIKDVHHTIESNARLEFECIWSEHESTKTPRSILSDLLSNKINSLNDSIQTSSLWTDQSLRRKIISAACPKVLLNLLGVDKIMERVPEPYVKAIFGSYLASRFVYKYGLNSNEFAFYTYMETLKQQ

SEQ ID NO: 45 - DNA, 1203 bp, Escherichia coli
atgtcgagtaagttagtactggttctgaactgcggtagttcttcactgaaatttgccatcatcgatgcagtaaatggtgaagagtacctttctggtttagccgaatgtttccacctgcccgaagcacgtatcaaatggaaaatggacggcaataaacaggaagcggctttaggtgcaggcgccgctcacagcgaagcgctcaactttatcgttaatactattctggcacaaaaaccagaactgtctgcgcagctgactgctatcggtcaccgtatcgtacacggcggcgaaaagtataccagctccgtagtgatcgatgagtctgttattcagggtatcaaagatgcagcttcttttgcaccgctgcacaacccggctcacctgatcggtatcgaagaagctctgaaatctttcccacagctgaaagacaaaaacgttgctgtatttgacaccgcgttccaccagactatgccggaagagtcttacctctacgccctgccttacaacctgtacaaagagcacggcatccgtcgttacggcgcgcacggcaccagccacttctatgtaacccaggaagcggcaaaaatgctgaacaaaccggtagaagaactgaacatcatcacctgccacctgggcaacggtggttccgtttctgctatccgcaacggtaaatgcgttgacacctctatgggcctgaccccgctggaaggtctggtcatgggtacccgttctggtgatatcgatccggcgatcatcttccacctgcacgacaccctgggcatgagcgttgacgcaatcaacaaactgctgaccaaagagtctggcctgctgggtctgaccgaagtgaccagcgactgccgctatgttgaagacaactacgcgacgaaagaagacgcgaagcgcgcaatggacgtttactgccaccgcctggcgaaatacatcggtgcctacactgcgctgatggatggtcgtctggacgctgttgtattcactggtggtatcggtgaaaatgccgcaatggttcgtgaactgtctctgggcaaactgggcgtgctgggctttgaagttgatcatgaacgcaacctggctgcacgtttcggcaaatctggtttcatcaacaaagaaggtacccgtcctgcggtggttatcccaaccaacgaagaactggttatcgcgcaagacgcgagccgcctgactgcctga

SEQ ID NO: 46 - DNA, 2145 bp, Escherichia coli
gtgtcccgtattattatgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcgtcgcaaactaccacgctaacaccaaagacgctgaagtcgttctggttgaaggtctggtcccgacacgtaagcaccagtttgcccagtctctgaactacgaaatcgctaaaacgctgaatgcggaaatcgtcttcgttatgtctcagggcactgacaccccggaacagctgaaagagcgtatcgaactgacccgcaacagcttcggcggtgccaaaaacaccaacatcaccggcgttatcgttaacaaactgaacgcaccggttgatgaacagggtcgtactcgcccggatctgtccgagattttcgacgactcttccaaagctaaagtaaacaatgttgatccggcgaagctgcaagaatccagcccgctgccggttctcggcgctgtgccgtggagctttgacctgatcgcgactcgtgcgatcgatatggctcgccacctgaatgcgaccatcatcaacgaaggcgacatcaatactcgccgcgttaaatccgtcactttctgcgcacgcagcattccgcacatgctggagcacttccgtgccggttctctgctggtgacttccgcagaccgtcctgacgtgctggtggccgcttgcctggcagccatgaacggcgtagaaatcggtgccctgctgctgactggcggttacgaaatggacgcgcgcatttctaaactgtgcgaacgtgctttcgctaccggcctgccggtatttatggtgaacaccaacacctggcagacctctctgagcctgcagagcttcaacctggaagttccggttgacgatcacgaacgtatcgagaaagttcaggaatacgttgctaactacatcaacgctgactggatcgaatctctgactgccacttctgagcgcagccgtcgtctgtctccgcctgcgttccgttatcagctgactgaacttgcgcgcaaagcgggcaaacgtatcgtactgccggaaggtgacgaaccgcgtaccgttaaagcagccgctatctgtgctgaacgtggtatcgcaacttgcgtactgctgggtaatccggcagagatcaaccgtgttgcagcgtctcagggtgtagaactgggtgcagggattgaaatcgttgatccagaagtggttcgcgaaagctatgttggtcgtctggtcgaactgcgtaagaacaaaggcatgaccgaaaccgttgcccgcgaacagctggaagacaacgtggtgctcggtacgctgatgctggaacaggatgaagttgatggtctggtttccggtgctgttcacactaccgcaaacaccatccgtccgccgctgcagctgatcaaaactgcaccgggcagctccctggtatcttccgtgttcttcatgctgctgccggaacaggtttacgtttacggtgactgtgcgatcaacccggatccgaccgctgaacagctggcagaaatcgcgattcagtccgctgattccgctgcggccttcggtatcgaaccgcgcgttgctatgctctcctactccaccggtacttctggtgcaggtagcgacgtagaaaaagttcgcgaagcaactcgtctggcgcaggaaaaacgtcctgacctgatgatcgacggtccgctgcagtacgacgctgcggtaatggctgacgttgcgaaatccaaagcgccgaactctccggttgcaggtcgcgctaccgtgttcatcttcccggatctgaacaccggtaacaccacctacaaagcggtacagcgttctgccgacctgatctccatcgggccgatgctgcagggtatgcgcaagccggttaacgacctgtcccgtggcgcactggttgacgatatcgtctacaccatcgcgctgactgcgattcagtctgcacagcagcagtaa

SEQ ID NO: 47 - DNA, 2676 bp, Escherichia coli
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggtgaaactgcagcgaagaaagaagctgctccggctaaagctgagaaaaaagcgaaaaaatccgcttaa

SEQ ID NO: 48 - DNA, 1809 bp, Escherichia coli
gtgcaaacctttcaagccgatcttgccattgtaggcgccggtggcgcgggattacgtgctgcaattgctgccgcgcaggcaaatccgaatgcaaaaatcgcactaatctcaaaagtatacccgatgcgtagccataccgttgctgcagaagggggctccgccgctgtcgcgcaggatcatgacagcttcgaatatcactttcacgatacagtagcgggtggcgactggttgtgtgagcaggatgtcgtggattatttcgtccaccactgcccaaccgaaatgacccaactggaactgtggggatgcccatggagccgtcgcccggatggtagcgtcaacgtacgtcgcttcggcggcatgaaaatcgagcgcacctggttcgccgccgataagaccggcttccatatgctgcacacgctgttccagacctctctgcaattcccgcagatccagcgttttgacgaacatttcgtgctggatattctggttgatgatggtcatgttcgcggcctggtagcaatgaacatgatggaaggcacgctggtgcagatccgtgctaacgcggtcgttatggctactggcggtgcgggtcgcgtttatcgttacaacaccaacggcggcatcgttaccggtgacggtatgggtatggcgctaagccacggcgttccgctgcgtgacatggaattcgttcagtatcacccaaccggtctgccaggttccggtatcctgatgaccgaaggttgccgcggtgaaggcggtattctggtcaacaaaaatggctaccgttatctgcaagattacggcatgggcccggaaactccgctgggcgagccgaaaaacaaatatatggaactgggtccacgcgacaaagtctctcaggccttctggcacgaatggcgtaaaggcaacaccatctccacgccgcgtggcgatgtggtttatctcgacttgcgtcacctcggcgagaaaaaactgcatgaacgtctgccgttcatctgcgaactggcgaaagcgtacgttggcgtcgatccggttaaagaaccgattccggtacgtccgaccgcacactacaccatgggcggtatcgaaaccgatcagaactgtgaaacccgcattaaaggtctgttcgccgtgggtgaatgttcctctgttggtctgcacggtgcaaaccgtctgggttctaactccctggcggaactggtggtcttcggccgtctggccggtgaacaagcgacagagcgtgcagcaactgccggtaatggcaacgaagcggcaattgaagcgcaggcagctggcgttgaacaacgtctgaaagatctggttaaccaggatggcggcgaaaactgggcgaagatccgcgacgaaatgggcctggctatggaagaaggctgcggtatctaccgtacgccggaactgatgcagaaaaccatcgacaagctggcagagctgcaggaacgcttcaagcgcgtgcgcatcaccgacacttccagcgtgttcaacaccgacctgctctacaccattgaactgggccacggtctgaacgttgctgaatgtatggcgcactccgcaatggcacgtaaagagtcccgcggcgcgcaccagcgtctggacgaaggttgcaccgagcgtgacgacgtcaacttcctcaaacacaccctcgccttccgcgatgctgatggcacgactcgcctggagtacagcgacgtgaagattactacgctgccgccagctaaacgcgtttacggtggcgaagcggatgcagccgataaggcggaagcagccaataagaaggagaaggcgaatggctga

SEQ ID NO: 49 - DNA, 735 bp, Escherichia coli
atggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggttcctgcggcatgatggttaacaacgtgccaaaactggcatgtaaaaccttcctgcgtgattacaccgacggtatgaaggttgaagcgttagctaacttcccgattgaacgcgatctggtggtcgatatgacccacttcatcgaaagtctggaagcgatcaaaccgtacatcatcggcaactcccgcaccgcggatcagggtactaacatccagaccccggcgcagatggcgaagtatcaccagttctccggttgcatcaactgtggtttgtgctacgccgcgtgcccgcagtttggcctgaacccagagttcatcggtccggctgccattacgctggcgcatcgttataacgaagatagccgcgaccacggtaagaaggagcgtatggcgcagttgaacagccagaacggcgtatggagctgtactttcgtgggctactgctccgaagtctgcccgaaacacgtcgatccggctgcggccattcagcagggcaaagtagaaagttcgaaagactttcttatcgcgaccctgaaaccacgctaa

SEQ ID NO: 50 - DNA, 396 bp, Escherichia coli
atgacgactaaacgtaaaccgtatgtacggccaatgacgtccacctggtggaaaaaattgccgttttatcgcttttacatgctgcgcgaaggcacggcggttccggctgtgtggttcagcattgaactgattttcgggctgtttgccctgaaaaatggcccggaagcctgggcgggattcgtcgactttttacaaaacccggttatcgtgatcattaacctgatcactctggcggcagctctgctgcacaccaaaacctggtttgaactggcaccgaaagcggccaatatcattgtaaaagacgaaaaaatgggaccagagccaattatcaaaagtctctgggcggtaactgtggttgccaccatcgtaatcctgtttgttgccctgtactggtaa

SEQ ID NO: 51 - DNA, 360 bp, Escherichia coli
atgattaatccaaatccaaagcgttctgacgaaccggtattctggggcctcttcggggccggtggtatgtggagcgccatcattgcgccggtgatgatcctgctggtgggtattctgctgccactggggttgtttccgggtgatgcgctgagctacgagcgcgttctggcgttcgcgcagagcttcattggtcgcgtattcctgttcctgatgatcgttctgccgctgtggtgtggtttacaccgtatgcaccacgcgatgcacgatctgaaaatccacgtacctgcgggcaaatgggttttctacggtctggctgctatcctgacagttgtcacgctgattggtgtcgttacaatctaa

SEQ ID NO: 52 - DNA, 990 bp, Escherichia coli
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggtttaa

SEQ ID NO: 53 - DNA, 459 bp, Escherichia coli
atggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaa

SEQ ID NO: 54 - DNA, 741 bp, Escherichia coli
atgtcagttattggtcgcattcactcctttgaatcctgtggaaccgtagacggcccaggtattcgctttatcacctttttccagggctgcctgatgcgctgcctgtattgtcataaccgcgacacctgggacacgcatggcggtaaagaagttaccgttgaagatttgatgaaggaagtggtgacctatcgccactttatgaacgcttccggcggcggcgttaccgcatccggcggtgaagcaatcctgcaagctgagtttgttcgtgactggttccgcgcctgcaaaaaagaaggcattcatacctgtctggacaccaacggttttgttcgtcgttacgatccggtgattgatgaactgctggaagtaaccgacctggtaatgctcgatctcaaacagatgaacgacgagatccaccaaaatctggttggagtttccaaccaccgcacgctggagttcgctaaatatctggcgaacaaaaatgtgaaggtgtggatccgctacgttgttgtcccaggctggtctgacgatgacgattcagcgcatcgcctcggtgaatttacccgtgatatgggcaacgttgagaaaatcgagcttctcccctaccacgagctgggcaaacacaaatgggtggcaatgggtgaagagtacaaactcgacggtgttaaaccaccgaagaaagagaccatggaacgcgtgaaaggcattcttgagcagtacggtcataaggtaatgttctaa

SEQ ID NO: 55 - DNA, 2283 bp, Escherichia coli
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaacgggtaacaccccagacggtcgtcgtgctggcgcgccgttcggaccgggtgctaacccgatgcacggtcgtgaccagaaaggtgcagtagcctctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaa

SEQ ID NO: 56 - DNA, 1302 bp, Escherichia coli
atgctagttgtagaactcatcatagttttgctggcgatcttcttgggcgccagattggggggaataggtattggttttgcaggcggattgggggtgctggttcttgccgctattggcgttaaacccggtaacatcccgttcgatgtcatctccattatcatggcggttatcgccgctatttctgccatgcaggttgctggcggtctggactatctggttcatcagacagaaaagctgctgcgccgtaacccgaaatacatcacgatcctcgcaccgatcgtgacctatttcctgactatctttgctggtactggcaacatctctctggcgacactgccagttatcgctgaagttgcgaaggaacaaggcgttaaaccttgccgtccgctgtctactgcagtggtatccgcgcagattgcgatcaccgcatcgccaatctcagcggcagtggtttacatgtcttccgtgatggaaggtcatggcatcagctacctccatctgctctccgtggtcatcccgtccaccctgctggcggttctggtgatgtccttcctggtcactatgctgttcaactccaaactctctgacgatccgatttatcgcaagcgtctggaagagggcctggttgaactgcgcggtgaaaagcagattgaaatcaaatccggtgcaaaaacgtccgtctggctgttcctgctgggcgtagttggcgtggttatctatgcaatcatcaacagcccaagcatgggtctggttgaaaaaccgctgatgaacaccaccaacgcaatcctgatcatcatgctcagcgttgcaactctgaccaccgttatctgtaaagtcgataccgacaacatcctcaactccagcaccttcaaagcaggtatgagcgcctgtatttgtatcctgggtgttgcgtggctgggcgatactttcgtttccaacaacatcgactggatcaaagataccgctggtgaagtgattcagggtcatccgtggctgctggccgtcatcttcttctttgcttctgctctgctgtactctcaggctgcaaccgcaaaagcactgatgccgatggctctggcactgaacgtttcaccgctgaccgctgttgcttctttcgctgcggtgtctggtctgttcattctgccgacctacccgacgctggttgctgcggtacagatggatgacacgggtactacccgtatcggtaaattcgtcttcaaccatccgttcttcatcccgggtactctgggtgttgccctggccgtttgcttcggcttcgtgctgggtagcttcatgctgtaa

SEQ ID NO: 57 - DNA, 1437 bp, Escherichia coli
atgtcaaacaacattcgtatcgaagaagatctgttgggtaccagggaagttccagctgatgcctactatggtgttcacactctgagagcgattgaaaacttctatatcagcaacaacaaaatcagtgatattcctgaatttgttcgcggtatggtaatggttaaaaaagccgcagctatggcaaacaaagagctgcaaaccattcctaaaagtgtagcgaatgccatcattgccgcatgtgatgaagtcctgaacaacggaaaatgcatggatcagttcccggtagacgtctaccagggcggcgcaggtacttccgtaaacatgaacaccaacgaagtgctggccaatatcggtctggaactgatgggtcaccaaaaaggtgaatatcagtacctgaacccgaacgaccatgttaacaaatgtcagtccactaacgacgcctacccgaccggtttccgtatcgcagtttactcttccctgattaagctggtagatgcgattaaccaactgcgtgaaggctttgaacgtaaagctgtcgaattccaggacatcctgaaaatgggtcgtacccagctgcaggacgcagtaccgatgaccctcggtcaggaattccgcgctttcagcatcctgctgaaagaagaagtgaaaaacatccaacgtaccgctgaactgctgctggaagttaaccttggtgcaacagcaatcggtactggtctgaacacgccgaaagagtactctccgctggcagtgaaaaaactggctgaagttactggcttcccatgcgtaccggctgaagacctgatcgaagcgacctctgactgcggcgcttatgttatggttcacggcgcgctgaaacgcctggctgtgaagatgtccaaaatctgtaacgacctgcgcttgctctcttcaggcccacgtgccggcctgaacgagatcaacctgccggaactgcaggcgggctcttccatcatgccagctaaagtaaacccggttgttccggaagtggttaaccaggtatgcttcaaagtcatcggtaacgacaccactgttaccatggcagcagaagcaggtcagctgcagttgaacgttatggagccggtcattggccaggccatgttcgaatccgttcacattctgaccaacgcttgctacaacctgctggaaaaatgcattaacggcatcactgctaacaaagaagtgtgcgaaggttacgtttacaactctatcggtatcgttacttacctgaacccgttcatcggtcaccacaacggtgacatcgtgggtaaaatctgtgccgaaaccggtaagagtgtacgtgaagtcgttctggaacgcggtctgttgactgaagcggaacttgacgatattttctccgtacagaatctgatgcacccggcttacaaagcaaaacgctatactgatgaaagcgaacagtaa

SEQ ID NO: 58 - DNA, 2461 bp, Halomonas elongata
atgaacgcaaccacagagccctttacaccctccgccgacctggccaagcccagcgtggccgatgccgtggtcggccatgaggcctcaccgctcttcatccgcaagccaagccccgatgacggctggggcatctacgagctggtcaagtcctgtccgcctctcgacgtcaattccgcctacgcctatctgttgctggccacccagttccgcgatagctgcgccgtggcgaccaacgaagagggcgagatcgtcggcttcgtttccggctacgtgaagagcaacgcccccgatacctatttcctctggcaggttgccgtgggcgagaaggcacgtggcaccggcctggcccgtcgtctggtggaagccgtgatgacacgcccggaaatggccgaggtccaccatctcgagaccactatcacgcccgacaaccaggcgtcctggggcttgttccgccgtctcgccgatcgctggcaggcgccgttgaacagccgcgaatacttctccaccgatcaactcggcggtgagcatgacccggaaaacctcgttcgcatcggcccgttccagaccgaccagatctgagccgggacgccgcctggccggcccggtacgggccggcaacccgtcttttcgttttatcactttcccccacaggaggtcgcaatgcagacccagattctcgaacgcatggagtccgacgttcggacctactcccgctccttcccggtcgtcttcaccaaggcgcgcaatgcccgcctgaccgacgaggaagggcgcgagtacatcgacttcctggccggtgccggcaccctgaactacggccacaacaacccgcacctcaagcaggcgctgctcgactatatcgacagcgacggcatcgtccacggcctggacttctggactgcggccaagcgcgactatctggaaaccctggaagaggtgatcctcaagccgcgcggtctcgactacaaggtgcatctgcccggaccgactggcaccaacgccgtcgaggcggccattcgcctggcccgggtcgccaaggggcgccacaatatcgtctccttcaccaacggctttcatggcgtcaccatgggcgcgctggcgaccaccggtaaccgcaagttccgcgaggccaccggtggcgtgccgacccaggctgcttccttcatgccgttcgatggctacctcggcagcagcaccgacaccctcgactacttcgagaagctgctcggcgacaagtccggcggcctggacgtgcccgcggcggtgatcgtcgagacagtgcagggcgagggcggtatcaatgtcgccggcctggagtggctcaagcgcctcgagagcatctgccgcgccaatgacatcctgctgatcatcgacgacatccaggcgggctgcggccggaccggcaagttcttcagcttcgagcatgccggcatcacgccggatatcgtgaccaactccaagtcgctgtccggttacggcctgccgttcgctcacgtcctgatgcgccccgagctcgacaagtggaagcccggtcagtacaacggcaccttccgcggcttcaacctggctttcgccactgctgctgccgccatgcgcaagtactggagcgacgacaccttcgagcgtgacgtgcagcgcaaggctcgcatcgtcgaggaacgcttcggcaagatcgccgcctggctgagcgagaacggcatcgaggcctccgagcgcggccgcgggctgatgcggggcatcgacgtgggttccggcgatatcgccgacaagatcacccaccaagccttcgagaacgggttgatcatcgaaaccagcggtcaggacggcgaagtggtcaagtgcctgtgcccgctgaccattcccgacgaagacctggtcgagggactcgacatcctcgagaccagcaccaagcaggcctttagctgatcgcctgaggtgcgccatcgggcctgtccatggcatcctgtatcggtcggccgtgcgcggccggccagtcattgattcactggagaatcgacatgatcgttcgcaatctcgaagaagcgcgccagaccgaccgtctggtcaccgccgaaaacggcaactgggacagcacccgcctgtcgctggccgaagatggtggcaactgctccttccacatcacccgcatcttcgagggtaccgagacccacatccactataagcatcacttcgaggctgtttattgcatcgaaggcgagggcgaagtggaaaccctggccgatggcaagatctggcccatcaagccgggtgacatctacatcctcgaccagcacgacgagcacctgctgcgcgccagcaagaccatgcacctggcctgcgtgttcacgccgggcctgaccggcaacgaagtgcaccgcgaagacggttcctacgcacctgccgacgaagccgacgaccagaagccgctgtaacccggcgcagtattctgccgtctcgcacg

SEQ ID NO: 59 - DNA, 1275 bp, Bacillus subtilis
atgtcagcaaagcaagtctcgaaagatgaagaaaaagaagctcttaacttatttctgtctacccaaacaatcattaaggaagcccttcggaagctgggttatccgggagatatgtatgaactcatgaaagagccgcagagaatgctcactgtccgcattccggtcaaaatggacaatgggagcgtcaaagtgttcacaggctaccggtcacagcacaatgatgctgtcggtccgacaaaggggggcgttcgcttccatccagaagttaatgaagaggaagtaaaggcattatccatttggatgacgctcaaatgcgggattgccaatcttccttacggcggcgggaagggcggtattatttgtgatccgcggacaatgtcatttggagaactggaaaggctgagcagggggtatgtccgtgccatcagccagatcgtcggtccgacaaaggatattccagctcccgatgtgtacaccaattcgcagattatggcgtggatgatggatgagtacagccggctgcgggaattcgattctccgggctttattacaggtaaaccgcttgttttgggaggatcgcaaggacgggaaacagcgacggcacagggcgtcacgatttgtattgaagaggcggtgaagaaaaaagggatcaagctgcaaaacgcgcgcatcatcatacagggctttggaaacgcgggtagcttcctggccaaattcatgcacgatgcgggcgcgaaggtgatcgggatttctgatgccaatggcgggctctacaacccagacggccttgatatcccttatttgctcgataaacgggacagctttggtatggtcaccaatttatttactgacgtcatcacaaatgaggagctgcttgaaaaggattgcgatattttagtgcctgccgcgatctccaatcaaatcacagccaaaaacgcacataacattcaggcgtcaatcgtcgttgaagcggcgaacggcccgacaaccattgatgccactaagatcctgaatgaaagaggcgtgctgcttgtgccggatatcctagcgagtgccggcggcgtcacggtttcttattttgaatgggtgcaaaacaaccaaggatattattggtcggaagaagaggttgcagaaaaactgagaagcgtcatggtcagctcgttcgaaacaatttatcaaacagcggcaacacataaagtggatatgcgtttggcggcttacatgacgggcatcagaaaatcggcagaagcatcgcgtttccgcggatgggtctaa

SEQ ID NO: 60 - DNA, 1599 bp, Anaerobiospirillum succiniciproducens
atgagcttatctgaaagtttagctaaatacggcattactggtgctaccaacatcgtccacaatccttctcacgaggagttgtttgctgctgagactcaggcttctttagaaggcttcgagaagggcactgtaaccgagatgggtgcagtaaacgttatgactggcgtttacaccggccgttcccctaaggacaagttcattgttaagaacgaggcctccaaggagatctggtggacttctgatgagttcaagaacgacaacaagccagttaccgaagaggcatgggctcagttaaaggctttagcaggcaaggagctctctaacaagcctttatacgttgttgatctcttctgtggtgctaacgagaacacccgtctgaagatccgtttcgttatggaagtagcttggcaggctcacttcgtaaccaacatgttcatccgtcctactgaggaagagttaaagggcttcgagccagacttcgtagttctgaacgcttcaaaggctaaggttgagaacttcaaggagttaggcctcaactctgagactgctgttgtattcaacctcgcagagaagatgcagatcatcctcaacacctggtacggtggtgagatgaagaagggcatgttctccatgatgaacttctacttaccattacagggcatcgctgcaatgcactgctctgctaacaccgacctcgagggcaagaacactgctatcttcttcggtctgtcaggcactggtaagaccaccttatctaccgatcctaagcgtttactcatcggtgacgacgagcacggttgggacgatgatggcgtattcaacttcgaaggcggctgctacgctaaggttattaacctctctaaggaaaacgagccagacatctggggcgctatcaagcgtaacgctttattagagaacgtaactgttgatgctaacggcaaggttgactttgcagacaagtctgtaactgagaacacccgtgtttcttacccaatcttccacattaaaaacatcgttaagccagtttctaaggctccagctgctaagcgcgtaatcttcttatctgctgatgctttcggtgtattacctccagtatctatcctttctaaggagcagactaagtactacttcctctctggcttcaccgctaagttagctggtaccgagcgtggcattaccgagcctaccccaaccttctcttcatgcttcggtgctgctttcttaaccttacctccaaccaagtacgctgaagttctggttaagagaatggaagcttccggcgctaaggcttacttagtaaacactggctggaacggcactggcaagcgtatctccattaaggatacccgtggcattatcgacgcaatcttagacggttctatcgatactgcaaacaccgcaaccatcccttacttcaacttcaccgttcctactgagttaaagggcgttgacaccaagattctggacccacgtaacacctacgctgacgcttcagagtgggaagtaaaggctaaggacttagctgagcgcttccagaagaacttcaagaagttcgagtctttaggtggtgacttagttaaggctggtccacagttataa

SEQ ID NO: 61 - DNA, 1284 bp, Escherichia coli
atggctgatacaaaagcaaaactcaccctcaacggggatacagctgttgaactggatgtgctgaaaggcacgctgggtcaagatgttattgatatccgtactctcggttcaaaaggtgtgttcacctttgacccaggcttcacttcaaccgcatcctgcgaatctaaaattacttttattgatggtgatgaaggtattttgctgcaccgcggtttcccgatcgatcagctggcgaccgattctaactacctggaagtttgttacatcctgctgaatggtgaaaaaccgactcaggaacagtatgacgaatttaaaactacggtgacccgtcataccatgatccacgagcagattacccgtctgttccatgctttccgtcgcgactcgcatccaatggcagtcatgtgtggtattaccggcgcgctggcggcgttctatcacgactcgctggatgttaacaatcctcgtcaccgtgaaattgccgcgttccgcctgctgtcgaaaatgccgaccatggccgcgatgtgttacaagtattccattggtcagccatttgtttacccgcgcaacgatctctcctacgccggtaacttcctgaatatgatgttctccacgccgtgcgaaccgtatgaagttaatccgattctggaacgtgctatggaccgtattctgatcctgcacgctgaccatgaacagaacgcctctacctccaccgtgcgtaccgctggctcttcgggtgcgaacccgtttgcctgtatcgcagcaggtattgcttcactgtggggacctgcgcacggcggtgctaacgaagcggcgctgaaaatgctggaagaaatcagctccgttaaacacattccggaatttgttcgtcgtgcgaaagacaaaaatgattctttccgcctgatgggcttcggtcaccgcgtgtacaaaaattacgacccgcgcgccaccgtaatgcgtgaaacctgccatgaagtgctgaaagagctgggcacgaaggatgacctgctggaagtggctatggagctggaaaacatcgcgctgaacgacccgtactttatcgagaagaaactgtacccgaacgtcgatttctactctggtatcatcctgaaagcgatgggtattccgtcttccatgttcaccgtcattttcgcaatggcacgtaccgttggctggatcgcccactggagcgaaatgcacagtgacggtatgaagattgcccgtccgcgtcagctgtatacaggatatgaaaaacgcgactttaaaagcgatatcaagcgttaa